(19) United States
(12) Patent Application Publication
(10) Pub. No.: US 2004/0115737 A1
(43) Pub. Date: Jun. 17, 2004

(54) Cd36 as a heat shock protein receptor and uses thereof

(76) Inventors: Naveed Panjwani, Slough (GB); James Zabrecky, Waltham, MA (US)

Correspondence Address: Pennie & Edmonds, 1155 Avenue of the Americas, New York, NY, 10036-2711, US

(21) Appl. No.: 10/398,593

(22) PCT Filed: Oct. 5, 2001
(86) PCT No.: PCT/US01/31401

(51) Int. Cl. G01N 33/53; G01N 33/567

(52) U.S. Cl. 435/7.2

(57) ABSTRACT

The present invention relates to the use of CD36 (“CD36”) receptor as a heat shock protein receptor, cells that express CD36 bound to an HSP, and antibodies and other molecules that bind CD36-HSP complex. The invention also relates to screening assays to identify compounds that interact with CD36, and modulate the interaction of CD36 with its ligand, such as HSPs, and methods for using compositions comprising CD36-receptor sequences for the diagnosis and treatment of immune disorders, proliferative disorders, and infectious diseases.

This application claims benefit of U.S. patent application No. 60/238,865 filed Oct. 6, 2000, which is incorporated by reference herein in its entirety.

### 1. INTRODUCTION

The present invention relates to the use of CD36 as a heat shock protein receptor, cells that express CD36 bound to an HSP, and antibodies and other molecules that bind the CD36-HSP complex. The invention also relates to screening assays to identify compounds that modulate the interaction of an HSP with CD36, and methods for using compositions comprising CD36-receptor sequences for the diagnosis and treatment of immune disorders, proliferative disorders, and infectious diseases.

### 2. BACKGROUND OF THE INVENTION

2.1. Heat Shock Proteins

Heat shock proteins (HSPs), also referred to as stress proteins, were first identified as proteins synthesized by cells in response to heat shock. Hsps have classified into five families, based on molecular weight, Hsp100, Hsp90, Hsp70, Hsp60, and smHsp. Many members of these families were found subsequently to be induced in response to other stressful stimuli including nutrient deprivation, metabolic disruption, oxygen radicals, and infection with intracellular pathogens (see Welch, May 1993, Scientific American 56-64; Young, 1990, Annu. Rev. Immunol. 8:401-420; Craig, 1993, Science 260:1902-1903; Gething et al., 1992, Nature 355:33-45; and Lindquist et al., 1988, Annu. Rev. Genetics 22:631-677).

Heat shock proteins are among the most highly conserved proteins in existence. For example, DnaK, the Hsp70 from E. coli has about 50% amino acid sequence identity with Hsp70 proteins from excoriates (Bardwell et al., 1984, Proc. Natl. Acad. Sci. 81:848-852).

The Hsp60 and Hsp90 families also show similarly high levels of intra-family conservation (Hickey et al., 1989, Mol. Cell. Biol. 9:2615-2626; Jindal, 1989, Mol. Cell. Biol. 9:2279-2283). In addition, it has been discovered that the Hsp60, Hsp70 and Hsp90 families are composed of proteins that are related to the stress proteins in sequence, for example, having greater than 35% amino acid identity, but whose expression levels are not altered by stress.

Studies on the cellular response to heat shock and other physiological stresses revealed that the HSPs are involved not only in cellular protection against these adverse conditions, but also in essential biochemical and immunological processes in unstressed cells. HSPs accomplish different kinds of chaperoning functions. For example, members of the Hsp70 family, located in the cell cytoplasm, nucleus, mitochondria, or endoplasmic reticulum (Lindquist et al., 1988, Ann. Rev. Genetics 22:631-677), are involved in the presentation of antigens to the cells of the immune system, and are also involved in the transfer, folding and assembly of proteins in normal cells. HSPs are capable of binding proteins or peptides, and releasing the bound proteins or peptides in the presence of adenosine triphosphate (ATP) or low pH.

2.2. Immunogenicity of HSP-Peptide Complexes

Srivastava et al. demonstrated immune response to methylcholanthrene-induced sarcomas of inbred mice (1988, Immunol. Today 9:78-83). In these studies, it was found that the molecules responsible for the individually distinct immunogenicity of these tumors were glycoproteins of 96 kDa (gp96) and intracellular proteins of 84 to 86 kDa (Srivastava et al., 1986, Proc. Natl. Acad. Sci. USA 83:3407-3411; Ullrich et al., 1986, Proc. Natl. Acad. Sci. USA 83:3121-3125). Immunization of mice with gp96 or p84/86 isolated from a particular tumor rendered the mice immune to that particular tumor, but not to antigenically distinct tumors. Isolation and characterization of genes encoding gp96 and p84/86 revealed significant homology between them, and showed that gp96 and p84/86 were, respectively, the endoplasmic reticular and cytosolic counterparts of the same heat shock proteins (Srivastava et al., 1988, Immunogenetics 28:205-207; Srivastava et al., 1991, Curr. Top. Microbiol. Immunol. 167:109-123). Further, Hsp70 was shown to elicit immunity to the tumor from which it was isolated but not to antigenically distinct tumors. However, Hsp70 depleted of peptides was found to lose its immunogenic activity (Udono and Srivastava, 1993, J. Exp. Med. 178:1391-1396). These observations suggested that the heat shock proteins are not immunogenic per se, but form noncovalent complexes with antigenic peptides, and the complexes can elicit specific immunity to the antigenic peptides (Srivastava, 1993, Adv. Cancer Res. 62:153-177; Udono et al., 1994, J. Immunol., 152:5398-5403; Suto et al., 1995, Science, 269:1585-1588).

Noncovalent complexes of HSPs and peptide, purified from cancer cells, can be used for the treatment and prevention of cancer and have been described in PCT publications WO 96/10411, dated Apr. 11, 1996, and WO 97/10001, dated Mar. 20, 1997 (U.S. Pat. No. 5,750,119 issued Apr. 12, 1998, and U.S. Pat. No. 5,837,251 issued Nov. 17, 1998, respectively, each of which is incorporated by reference herein in its entirety). The isolation and purification of stress protein-peptide complexes has been described, for example, from pathogen-infected cells, and can be used for the treatment and prevention of infection caused by the pathogen, such as viruses, and other intracellular pathogens, including bacteria, protozoa, fingi and parasites (see, for example, PCT Publication WO 95/24923, dated Sep. 21, 1995). Immunogenic stress protein-peptide complexes can also be prepared by in vitro complexing of stress protein and antigenic peptides, and the uses of such complexes for the treatment and prevention of cancer and infectious diseases has been described in PCT publication WO 97/10000, dated Mar. 20, 1997 (U.S. Pat. No. 6,030,618 issued Feb. 29, 2000. The use of stress protein-peptide complexes for sensitizing antigen presenting cells in vitro for use in adoptive immunotherapy is described in PCT publication WO 97/10002, dated Mar. 20, 1997 (see also U.S. Pat. No. 5,985,270 issued Nov. 16, 1999).

2.3. CD36

CD36 is a member of the class B scavenger receptor family and is primarily expressed in capillary endothelial cells, mammary secretory epithelial cells, differentiated adipose cells, B cells, macrophages, and several types of tumour cells. CD36 is believed to play a role in platelet adhesion and aggregation, phagocytosis of apoptotic cells, and in the metabolism of long-chain fatty acids. CD36 is located in the plasma membrane in microdomains called caveolae, which have been implicated in cellular transport and signaling pathways. Structurally, CD36 has a large (463 aa) extracellular domain, a single transmembrane domain and a short cytoplasmic tail containing a putative tyrosine kinase docking site. Amino acid residues #184-204 of CD36 make up a hydrophobic stretch of the receptor that is most likely associated with the plasma membrane. Anti-CD36 isoantibody recognizes a highly antigenic structure at amino acids 155-183 which is known as the immunodominant domain.

CD36 is expressed in both the monocytic and megakaryocytic lineage where it is upregulated during differentiation. Monocyte expression of CD36 is regulated by M-CSF and IL-4 and through adherence to activated endothelial cells (Yesner et al., 1996, Arterioscler. Thromb. Vasc. Biol. 16:1019-1025; Huh et al., 1995, J. Biol. Chem. 270:6267-6271). Expression of murine or human CD36 in CD36-deficient cells results in specific and high-affinity binding of oxidized LDL, followed by LDL internalization and degradation (Endemann et al., 1993, J. Biol. Chem. 268:11811-11816; Navazo et. al., 1996, Arterioscler. Thromb. Vasc. Biol. 16:1033-1039). CD36 also binds to long-chain fatty acids and its expression has been shown to be upregulated in endothelial cells in tissues involved in fatty-acid transport and metabolism (Greenwalt et al., 1995, J. Clin. Invest. 96:1382-1388).

CD36 deficiency in humans is seen in approximately 3% of the Japanese population and 0.3% of Caucasians. Afflicted people are phenotypically normal but some subjects suffer from a reduced capacity to bind and internalize oxidized LDL (Nozaki et al., 1995, J. Clin. Invest. 96:1859-1865).

CD36 has also been shown to play a role in cytoadherence to Plasmodium falciparum-infected erythrocytes. After infection, P.falciparum become sequestered within the microvasculature, which helps contribute to the survival of the bacterium by preventing clearance in the spleen. CD36 binds to the PfEMP1 protein which is produced by P.falciparum in the infected erythrocyte. This binding can induce an oxidative burst of monocytes and platelet activation (Okenhouse et al., 1989, J. Clin. Invest. 84:468-475; Huang et. al., 1991, Proc. Natl. Acad. Sci. USA 88:7844-7848).

2.4. GP96

Major histocompatibility complex (MHC) molecules present antigens on the cell surface of antigen-presenting cells. Cytotoxic T lymphocytes (CTLs) then recognize MHC molecules and their associated peptides and kill the target cell. Antigens are processed by two distinct antigen processing routes depending upon whether their origin is intracellular or extracellular. Intracellular or endogenous protein antigens, i.e., antigens synthesized within the antigen-presenting cell, are presented by MHC class I (MHC I) molecules to CD8+ cytotoxic T lymphocytes. On the other hand, extracellular or exogenously synthesized antigenic determinants are presented on the cell surface of “specialized” or “professional” APCs (macrophages, for example) by MHC class II molecules to CD4+ T cells (see, generally, Fundamental Immunology, W. E. Paul (ed.), New York: Raven Press, 1984). This compartmental segregation of antigen processing routes is important to prevent tissue destruction that could otherwise occur during an immune response as a result of shedding of neighboring cell MHC I antigens.

The heat shock protein gp96 chaperones a wide array of peptides, depending upon the source from which gp96 is isolated (for review, see Srivastava et al., 1998, Immunity 8: 657-665). Tumor-derived gp96 carries tumor-antigenic peptides (Ishii et al., 1999, J. Immunology 162:1303-1309); gp96 preparations from virus-infected cells carry viral epitopes (Suto and Srivastava, 1995, Science 269:1585-1588; Nieland et al., 1998, Proc. Natl. Acad. Sci. USA 95:1800-1805), and gp96 preparations from cells transfected with model antigens such as ovalbumin or β-galactosidase are associated with the corresponding epitopes (Arnold et al., 1995, J. Exp. Med.182:885-889; Breloer et al., 1998, Eur. J. Immunol. 28:1016-1021). The association of gp96 with peptides occurs in vivo (Menoret and Srivastava, 1999, Biochem. Biophys. Research Commun. 262:813-818). Gp96-peptide complexes, whether isolated from cells (Tamura et al., 1997, Science 278:117-120), or reconstituted in vitro (Blachere et al., 1997, J. Exp. Med. 186:1183-1406) are excellent immunogens and have been used extensively to elicit CD8+ T cell responses specific for the gp96-chaperoned antigenic peptides.

The capacity of gp96-peptide complexes to elicit an immune response is dependent upon the transfer of the peptide to MHC class I molecules of antigen-presenting cells (Suto and Srivastava, 1995, supra). Endogenously synthesized antigens chaperoned by gp96 in the endoplasmic reticulum [ER] can prime antigen-specific CD8+ T cells (or MHC I-restricted CTLs) in vivo; this priming of CD8+ T cells requires macrophages. However, the process whereby exogenously introduced gp96-peptide complexes elicit the antigen-specific CD8+ T cell response is not completely understood since there is no established pathway for the translocation of extracellular antigens into the class I presentation machinery. Yet antigenic peptides of extracellular origin associated with HSPs are somehow salvaged by macrophages, channeled into the endogenous pathway, and presented by MHC I molecules to be recognized by CD8+ lymphocytes (Suto and Srivastava, 1995, supra; Blachere et al., 1997, J. Exp. Med. 186:1315-22).

Several models have been proposed to explain the delivery of extracellular peptides for antigen presentation. One proposal, known as the “direct transfer” model, suggests that HSP-chaperoned peptides are transferred to MHC I molecules on the cell surface of macrophages for presentation to CD8+ T lymphocytes. Another suggestion is that soluble extracellular proteins can be trafficked to the cytosol via constitutive macropinocytosis in bone marrow-derived macrophages and dendritic cells (Norbury et al., 1997, Eur. J. Immunol. 27:280-288). Yet another proposed mechanism is that HSPs are taken up by the MHC class I molecules of the macrophage, which stimulate the appropriate T cells (Srivastava et al., 1994, Immunogenetics 39:93-98). Others have suggested that a novel intracellular trafficking pathway may be involved for the transport of peptides from the extracellular medium into the lumen of ER (Day et al., 1997, Proc. Natl. Acad. Sci. 94:8064-8069; Nicchitta, 1998, Curr. Opin. in Immunol. 10:103-109). Further suggestions include the involvement of phagocytes which (a) possess an ill-defined pathway to shunt protein from the phagosome into the cytosol where it would enter the normal class I pathway; (b) digest ingested material in lysosomes and regurgitate peptides for loading on the surface to class I molecules (Bevan, 1995, J. Exp. Med. 182:639-41).

Still others have proposed a receptor-mediated pathway for the delivery of extracellular peptides to the cell surface of APCs for antigen presentation. In view of the extremely small quantity of gp96-chaperoned antigenic peptides required for immunization (Blachere et al., 1997, supra), and the strict dependence of immunogenicity of gp96-peptide complexes on functional antigen presenting cells (APCs) (Udono et al., 1994, Proc. Natl. Acad. Sci. U.S.A. 91:3077-3081), APCs had been proposed to possess receptors for gp96 (Srivastava et al., 1994, Immunogenetics 39:93-98). Such a receptor was recently identified and determined to be the alpha (2) macroglobulin receptor or CD91 (Binder et al., 2000, Nature Immunol. 1:151-155). It has been proposed that gp96 carries peptides into a cell via CD91 and transfers these peptides to the MHC class I molecules of dendritic and antigen presenting cells.

The identification and characterization of specific molecules involved in HSP-mediated antigen presentation of peptides could provide useful reagents and techniques for eliciting specific immunity by HSP and HSP-peptide complexes, and for developing novel diagnostic and therapeutic methods.

Citation or discussion of a reference herein shall not be construed as an admission that such is prior art to the present invention.

### 3. SUMMARY OF THE INVENTION

The present invention relates to compositions and methods for the use of CD36 as a heat shock protein receptor. The invention is based, in part, on the Applicants discovery that CD36 is a cell surface receptor for heat shock proteins. In particular, the Applicants have shown herein that the heat shock protein gp96 binds to CD36 expressing cells, initiating signal transduction cascade, resulting in cytokine, chemokine, and nitric oxide production.

The present invention provides compositions comprising complexes of HSPs and CD36, and antibodies and other molecules that bind CD36 complex. The invention also encompasses methods for the use of CD36 as a heat shock protein receptor, including methods for screening for compounds that modulate the interaction of HSP and CD36, and methods for treatment and detection of HSP-CD36 mediated processes and HSP-CD36 related disorders and conditions, such as autoimmune disorders, proliferative disorders and infectious diseases.

The invention provides a method for identifying a compound that modulates an HSP-CD36 mediated process, comprising: (a) contacting a test compound with a heat shock protein and CD36; and (b) measuring the level of CD36 activity or expression, such that if the level of activity or expression measured in (b) differs from the level of CD36 activity in the absence of the test compound, then a compound that modulates an HSP-CD36 mediated process is identified. In one embodiment of this method the compound identified is an antagonist which interferes with the interaction of the heat shock protein with CD36, further comprising the step of: (c) determining whether the level interferes with the interaction of the heat shock protein and CD36. In another embodiment, the test compound is an antibody specific for CD36. In another embodiment, test compound is an antibody specific for a heat shock protein. In another embodiment, the test compound is a small molecule. In yet another embodiment, the test compound is a peptide. In another embodiment, the peptide comprises at least 5 consecutive amino acids of CD36. In yet another embodiment, the peptide comprises at least 5 consecutive amino acids of a heat shock protein sequence. In another embodiment, the compound is an agonist which enhances the interaction of the heat shock protein with CD36. In another embodiment, which the HSP-CD36 mediated process affects an autoimmune disorder, a disease or disorder involving cellular signaling, a disease or disorder involving cytokine clearance or inflammation, a proliferative disorder, a viral disorder or other infectious disease, hypercholesterolemia, Alzheimer's disease, diabetes, or osteoporosis;

The invention also provides a method for identifying a compound that modulates an HSP-CD36 mediated process, comprising: (a) contacting a test compound with a heat shock protein and a CD36-expressing cell; and (b) measuring the level of CD36 activity or expression in the cell, such that if the level of activity or expression measured in (b) differs from the level of CD36 activity in the absence of the test compound, then a compound that modulates an HSP-CD36 mediated process is identified. In yet another embodiment, the CD36 activity measured is the ability to interact with a heat shock protein.

The invention also encompasses a method for identifying a compound that modulates the binding of a heat shock protein to CD36, comprising: (a) contacting a heat shock protein with CD36, or fragment, or analog, derivative or mimetic thereof, in the presence of a test compound; and (b) measuring the amount of heat shock protein bound to CD36, or fragment, analog, derivative or mimetic thereof, such that if the amount of bound heat shock protein measured in (b) differs from the amount of bound heat shock protein measured in the absence of the test compound, then a compound that modulates the binding of an HSP to CD36 is identified. In another embodiment, CD36 contacted in step (a) is on a cell surface. In another embodiment, CD36 is immobilized to a solid surface. In another embodiment, the solid surface is a microtiter dish. In another embodiment, the amount of bound heat shock protein is measured by contacting the cell with a heat shock protein-specific antibody. In yet another embodiment, the heat shock protein is labeled and the amount of bound heat shock protein is measured by detecting the label. In another embodiment, the heat shock protein is labeled with a fluorescent label.

The invention further provides a method for identifying a compound that modulates heat shock protein-mediated cellular signaling by CD36-expressing cells comprising: (a) adding a test compound to a mixture of CD36-expressing cells consisting essentially of a heat shock protein associated with an antigenic molecule, under conditions conducive to CD36-mediated production of a signal transducer; (b) measuring the level of signal transducer by CD36-expressing cells, such that if the level measured in (b) differs from the level of said stimulation in the absence of the test compound, then a compound that modulates heat shock protein-mediated signal transducer stimulation by CD36-expressing cells is identified. In one embodiment of this method, the step of measuring the level of signal transducer stimulation of step (b) comprises: (i) adding CD36-expressing cells formed in step (a) to T cells under conditions conducive to signal transducer stimulation; and (ii) comparing the level of signal transducer activation of said T cells with the level of activation of T cells by a CD36-expressing cell formed in the absence of the test compound, wherein an increase or decrease in level of T cell activation indicates that a compound that modulates heat shock protein-mediated signal transduction by a CD36 expressing cells is identified.

In various embodiments, the heat shock protein used in the methods of the invention is gp96.

In another embodiment, the invention provides a method for detecting a heat shock protein-CD36 related disorder in a mammal comprising measuring the level of an HSP-CD36 mediated process in a patient sample, such that if the measured level differs from the level found in clinically normal individuals, then a heat shock CD36-related disorder is detected.

The invention also encompasses kits comprising compositions of the invention. In one embodiment, a kit is provided, packaged in one or more containers, comprising: (a) a purified heat shock protein, nucleic acid encoding a heat shock protein, or cell expressing a heat shock protein; and (b) a CD36 polypeptide, nucleic acid encoding a CD36 polypeptide, or cell expressing a CD36 polypeptide. In one embodiment, CD36 polypeptide, nucleic acid encoding CD36 polypeptide, or cell expressing CD36 polypeptide is purified. In another embodiment, the kit further comprises instructions for use in treating an autoimmune disorder, an infectious disease, or a proliferative disorder.

The invention also provides a method for modulating an immune response comprising administering to a mammal a purified compound that modulates the interaction of a heat shock protein with CD36. In one embodiment, the compound is an agonist which enhances the interaction of the heat shock protein and CD36. In another embodiment of this method the compound in an antagonist that interferes with the interaction between the heat shock protein and CD36.

The invention further provides a method for treating an autoimmune disorder comprising administering to a mammal in need of such treatment a purified compound that interferes with the interaction of a heat shock protein with CD36. In one embodiment of this method the compound in an antagonist that interferes with the interaction between the heat shock protein and CD36. In another embodiment, the antagonist is an antibody specific for CD36. In another embodiment, the antagonist is an antibody specific for a heat shock protein. In another embodiment, the antagonist is a small molecule. In another embodiment, the antagonist is a peptide. In another embodiment, the peptide comprises at least 5 consecutive amino acids of CD36. In another embodiment, the peptide comprises at least 5 consecutive amino acids of a heat shock protein sequence.

The invention further provides a method for increasing the immunopotency of a cancer cell or an infected cell comprising transforming said cell with a nucleic acid comprising a nucleotide sequence that (i) is operably linked to a promoter, and (ii) encodes a CD36 polypeptide.

Still further, the invention provides a method for increasing the immunopotency of a cancer cell or an infected cell comprising: (a) transforming said cell with a nucleic acid comprising a nucleotide sequence that (i) is operably linked to a promoter, and (ii) encodes a CD36 polypeptide, and (b) administering said cell to an individual in need of treatment, so as to obtain an elevated immune response.

The invention also provides a recombinant cancer cell transformed with a nucleic acid comprising a nucleotide sequence that (i) is operably linked to a promoter, and (ii) encodes a CD36 polypeptide. In one embodiment, the recombinant cell is a human cell.

In yet another embodiment, the invention provides a recombinant infected cell transformed with a nucleic acid comprising a nucleotide sequence that (i) is operably linked to a promoter, and (ii) encodes a CD36 polypeptide. In one embodiment, the recombinant cell is a human cell.

In another embodiment, the invention provides a method for screening for molecules that specifically bind CD36 comprising the steps of: (a) contacting CD36 with one or more test molecules under conditions conducive to binding; and (b) determining whether any of said test molecules specifically bind to CD36. In one embodiment of this method, test molecules are potential immunotherapeutic drugs.

The invention also provides a method for identifying a compound that modulates the binding of CD36 ligand to CD36 comprising: contacting CD36 with a CD36 ligand, or a CD36 binding fragment, analog, derivative, or mimetic thereof, in the presence of one or more test compound; and (b) measuring the amount of CD36 ligand, or fragment, analog, derivative or mimetic thereof, bound to CD36, such that if the amount of bound CD36 ligand measured in (b) differs from the amount of bound CD36 measured in the absence of the test compound, then a compound that modulates the binding of CD36 ligand to CD36 is identified.

In another embodiment, a method is provided for identifying a compound that modulates the interaction between CD36 and a CD36 ligand, comprising: (a) contacting CD36 with one or more test compounds and CD36 ligand; and (b) measuring the level of activity or expression, such that if the level of activity or expression measured in (b) differs from the level of CD36 activity in the absence of one or more test compounds, then a compound that modulates the interaction between CD36 and a CD36 ligand is identified.

In another embodiment, the invention provides a method for modulating an immune response comprising administering to a mammal a purified compound that binds to CD36 in an amount effective to modulate an immune response in the mammal.

In yet another embodiment, a method for treating or preventing a disease or disorder is provided comprising administering to a mammal a purified compound that binds to CD36 in an amount effective to treat or prevent a disease or disorder in the mammal. In one embodiment, the disease or disorder is cancer or an infectious disease.

In a further embodiment, a method is provided for treating an autoimmune disorder comprising administering to a mammal in need of such treatment a purified compound that binds to CD36 in an amount effective to treat an autoimmune disorder in the mammal.

The term “HSP-CD36 mediated process” as used herein refers to a process dependent and/or responsive, either directly or indirectly, to the interaction of HSP with CD36. Such processes include processes that result from an aberrant level of expression, synthesis and/or activity of CD36, such as signal transduction stimulating activities relating to the binding of the various CD36 ligands, including but not limited to, lipoprotein complexes, thrombospondin 1, P. falciparum erythrocyte membrane protein 1 (PfEMP1), LDL, and phospholipids. Such processes include, but are not limited to, phagocytosis of apoptotic cells and metabolism of long-chain fatty acids.

The terms “HSP-CD36 related disorder” and “HSP-CD36 related condition”, as used herein, refers to a disorder and a condition, respectively, involving a HSP-CD36 interaction. Such disorders and conditions may result, for example, from an aberrant ability of CD36 to interact with HSP, perhaps due to aberrant levels of HSP and/or CD36 expression, synthesis and/or activity relative to levels found in normal, unaffected, unimpaired individuals, levels found in clinically normal individuals, and/or levels found in a population whose levels represent a baseline, average HSP and/or CD36 levels. Such disorders include, but are not limited to, autoimmune disorders, diseases and disorders involving cellular signaling or growth disruption, diseases and disorders involving cytokine clearance and/or inflammation, proliferative disorders, viral disorders and other infectious diseases, hypercholesterolemia, Alzheimer's disease, diabetes, and osteoporosis.

The term “CD36 ligand” as used herein, refers to a molecule capable of binding to CD36. Such CD36 ligands include as well as known ligands, such as, but not limited to, lipoprotein complexes, thrombospondin 1, P. falciparum erythrocyte membrane protein 1 (PfEMP1), and phospholipids. In addition, CD36 ligands also include molecules which can readily be identified as CD36 ligands using standard binding assays well known in the art. Such CD36 ligands are typically endoctyosed by a cell upon binding to CD36.

### 4. BRIEF DESCRIPTION OF THE FIGURES

FIGS. 1A-E. Binding of fluorescent protein probes to CD36-expression gated populations of transiently transfected HEK293 cells. Shaded points and shaded bargraph represent CD36 expressing cells. Unshaded points and unshaded bargraph represent non-expressing and CD36^{low} cells. Horizontal axis represents protein concentration in micrograms/microliter and the vertical axis represents mean fluorescence intensity. A. Gp96-FITC. B. Histone-FITC. C. Acetylated low density lipoprotein-Dil (AcLDL-Dil). D. Ova-FITC. E. BSA(faf)-FITC.

FIGS. 2A-D. Comparison of MCP-1 chemokine production by CD36 null and wild type macrophages in response to various stimuli. Shaded points represent wild type macrophages and unshaded points represent CD36 null macrophages. Horizontal axis represents protein concentration in micrograms/milliliter in A, C, and D and endotoxin units/milliliter in B; vertical axis represents protein concentration picograms/milliliter. A. Macrophages incubated with native and boiled gp96. B. Macrophages incubated with native and boiled LPS. C. Macrophages incubated with native and boiled HSP70. D. Macrophages incubated with TNF-α and BSA.

FIGS. 3A-D. Comparison of MCP-1 chemokine production by CD36 null and wild type dendritic cells in response to various stimuli. Shaded points represent wild type macrophages and unshaded points represent CD36 null macrophages. Horizontal axis represents protein concentration in micrograms/milliliter in A, C, and D and endotoxin units/milliliter in B. A. Macrophages incubated with native and boiled gp96. B. Macrophages incubated with native and boiled LPS. C. Macrophages incubated with native and boiled HSP70. D. Macrophages incubated with TNF-α and BSA.

FIGS. 4A-D. Comparison of nitric oxide production by CD36 null and wild type macrophages in response to various stimuli. Shaded points represent wild type macrophages and unshaded points represent CD36 null macrophages. Horizontal axis represents protein concentration in micrograms/milliliter in A, C, and D and endotoxin units/milliliter in B. A. Macrophages incubated with native and boiled gp96. B. Macrophages incubated with native and boiled LPS. C. Macrophages incubated with native and boiled HSP70. D. Macrophages incubated with TNF-α and BSA.

FIGS. 5A-E. Comparison of nitric oxide production by CD36 null and wild type dendritic cells in response to various stimuli. Shaded points represent wild type macrophages and unshaded points represent CD36 null macrophages. Horizontal axis represents protein concentration in micrograms/milliliter in A, C, and D and endotoxin units/milliliter in B. A. Macrophages incubated with native and boiled gp96. B. Macrophages incubated with native and boiled LPS. C. Macrophages incubated with native and boiled HSP70. D. Macrophages incubated with TNF-a and BSA.

FIGS. 6A-C. The human CD36 cDNA (Genbank accession no. L06850) and predicted open reading frame of the CD36 protein (Genbank accession no. AAA16068).

FIGS. 7A-C. Increased binding of gp96 on CD36 expressing cells compared to non-expressing controls. Shaded points represent mock transfected cells and unshaded points represent CD36 transfected cells. The vertical axis represents mean fluorescence intensity and the horizontal axis represents protein concentration in micrograms/microliter. A. Gp96-FITC. B. Transferrin-FITC. C. Histone-FITC.

FIGS. 8A-D. Expression of CD36 transgene on cell surface of HEK293 transfectants and control cell lines. The horizontal axis represents fluorescence intensity and the vertical axis represents number of cells. A. HEK293 mock transfected cells. B. U937 cells (positive control). C. CD36 HECK293 transfected cells. D. CD36 HECK293 transfected cells.

FIG. 9. Expression of CD36 transgene on cell surface of HEK293 transfected cells and control cell lines. Horizontal axis represents fluorescence intensity and the vertical axis represents number of cells. The shaded area represents CD36 cells that are unstained. The first unshaded area furthest to the left represents CD36 wild type cells that are unstained. The middle unshaded area represents CD36 null cells stained with gp96-FITC. The unshaded area furthest to the right represents CD36 wild type cells stained with gp96-FITC.

FIGS. 10A-B. Analysis of blocking of gp-96-FITC binding by putative competitors. A. Shaded square represents PC93 mock transfected cells. Unshaded square represents CD36 transfected cells without pre-incubation. Unshaded triangle represents CD36 transfected cells preincubated with acetylated LDL. Unshaded circle represents CD36 transfected cells preincubated with oxidized LDL. B. Shaded square represents PC93 mock transfected cells. Unshaded square represents CD36 transfected cells without pre-incubation. Unshaded triangle represents CD36 transfected cells preincubated with anti-CD36 antibody. Unshaded circle represents CD36 transfected cells preincubated with anti-CD36 antibody.

### 5. DETAILED DESCRIPTION OF THE INVENTION

The present invention relates to compositions and methods for the use of CD36 as a heat shock protein (“HSP”) receptor. In particular, the present invention provides compositions comprising isolated CD36-ligand complexes, e.g., CD36-HSP complexes, including isolated and/or recombinant cells, and antibodies, molecules and compounds that modulate the interaction of CD36 with a CD36 ligand, such as HSP. The invention further encompasses methods for the use of CD36 as a heat shock protein receptor, including screening assays to identify compounds that modulate the interaction of CD36 with an HSP, or other CD36 ligand, and methods for the use of these molecules and complexes for the diagnosis and treatment of immune disorders, proliferative disorders, and infectious diseases.

The term “CD36 ligand” as used herein, refers to a molecule capable of binding to CD36. Such CD36 ligands include as well as known ligands, such as, but not limited to, lipoprotein complexes, thrombospondin 1, P. falciparum erythrocyte membrane protein 1 (PfEMP1), LDL, and phospholipids. In addition, CD36 ligands also include molecules which can readily be identified as CD36 ligands using standard binding assays well known in the art.

An HSP useful in the practice of the invention may be selected from among any cellular protein that satisfies the following criteria: the intracellular concentration of an HSP increases when a cell is exposed to a stressful stimulus; an HSP can bind other proteins or peptides, and can release the bound proteins or peptides in the presence of adenosine triphosphate (ATP) or low pH; or an HSP possesses at least 35% homology with any cellular protein having any of the above properties. Preferably, the HSP used in the compositions and methods of the present invention includes, but are not limited to, HSP90, gp96, BiP, Hsp70, DnaK, Hsc70, PhoE calreticulin, PDI, or an sHsp, alone or in combination.

In a preferred embodiment, an HSP is mammalian (e.g., mouse, rat, primate, domestic animal such as dog, cat, cow, horse), and is most preferably, human.

Hsps useful in the practice of the invention include, but are not limited to, members of the HSP60 family, HSP70 family, HSP90 family, HSP100 family, sHSP family, calreticulin, PDI, and other proteins in the endoplasmic reticulum that contain thioredoxin-like domain(s), such as, but not limited to, ERp72 and ERp61.

HSP analogs, muteins, derivatives, and fragments can also be used in place of HSPs according to the invention. An HSP peptide-binding “fragment” for use in the invention refers to a polypeptide comprising a HSP peptide-binding domain that is capable of becoming non-covalently associated with a peptide to form a complex that is capable of eliciting an immune response. In one embodiment, an HSP peptide-binding fragment is a polypeptide comprising an HSP peptide-binding domain of approximately 100 to 200 amino acids.

Databases can also be searched to identify sequences with various degrees of similarities to a query sequence using programs, such as FASTA and BLAST, which rank the similar sequences by alignment scores and statistics. Such nucleotide sequences of non-limiting examples of HSPs that can be used for preparation of the HSPs used in the methods of the invention are as follows: human Hsp70, Genbank Accession No. NM_{—}005345, Sargent et al., 1989, Proc. Natl. Acad. Sci. U.S.A., 86:1968-1972; human Hsp90, Genbank Accession No. X15183, Yamazaki et al., Nucl. Acids Res. 17:7108; human gp96: Genbank Accession No. X15187, Maki et al., 1990, Proc. Natl. Acad Sci., 87: 5658-5562; human BiP: Genbank Accession No. M19645; Ting et al., 1988, DNA 7: 275-286; human Hsp27, Genbank Accession No. M24743; Hickey et al., 1986, Nucleic Acids Res. 14:4127-45; mouse Hsp70: Genbank Accession No. M35021, Hunt et al., 1990, Gene, 87:199-204; mouse gp96: Genbank Accession No. M16370, Srivastava et al., 1987, Proc. Natl. Acad. Sci., 85:3807-3811; and mouse BiP: Genbank Accession No. U16277, Haas et al., 1988, Proc. Natl. Acad. Sci. U.S.A., 85: 2250-2254. Due to the degeneracy of the genetic code, the term “HSP sequence”, as used herein, refers not only to the naturally occurring amino acid and nucleotide sequence but also encompasses all the other degenerate sequences that encode the HSP.

The aforementioned HSP families also contain proteins that are related to HSPs in sequence, for example, having greater than 35% amino acid identity, but whose expression levels are not altered by stress. Therefore, it is contemplated that the definition of heat shock or stress protein, as used herein, embraces other proteins, mutants, analogs, and variants thereof having at least 35% to 55%, preferably 55% to 75%, and most preferably 75% to 85% amino acid identity with members of these families whose expression levels in a cell are enhanced in response to a stressful stimulus. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. USA 87:2264-2268, modified as in Karlin and Altschul, 1993, Proc. Natl. Acad. Sci. USA 90:5873-5877. Such an algorithm is incorporated into the NBLAST and XBLAST programs of Altschul, et al., 1990, J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al., 1997, Nucleic Acids Res.25:3389-3402. Alternatively, PSI-Blast can be used to perform an iterated search which detects distant relationships between molecules (Altschul et al., 1997, supra). When utilizing BLAST, Gapped BLAST, and PSI-Blast programs, the default parameters of the respective programs (e.g., XBLAST and NBLAST) can be used (see http://www.ncbi.nlm.nih.gov). Another preferred, non-limiting example of a mathematical algorithm utilized for the comparison of sequences is the algorithm of Myers and Miller, 1988, CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The immunogenic HSP-peptide complexes of the invention may include any complex containing an HSP and a peptide that is capable of inducing an immune response in a mammal. The peptides are preferably noncovalently associated with the HSP. Preferred complexes may include, but are not limited to, gp96-peptide complexes, HSP90-peptide complexes, HSP70-peptide complexes, HSP60-peptide complexes, HSP100-peptide complexes, calreticulin-peptide complexes, and sHSP-peptide complexes. For example, the HSP gp96 which is present in the endoplasmic reticulum of eukaryotic cells and is related to the cytoplasmic HSP90's can be used to generate an effective vaccine containing a gp96-peptide complex.

The HSPs, CD36, and/or antigenic molecules for use in the invention can be purified from natural sources, chemically synthesized, or recombinantly produced. Although the HSPs may be allogeneic to the patient, in a preferred embodiment, the HSPs are autologous to the patient to whom they are administered.

5.1 Compositions of the Invention

The present invention provides compositions that modulate the interaction between CD36 and a CD36 ligand, such as, for example, an HSP. Such compositions can be used in methods to elicit or modulate an immune response. Such compositions also include antibodies that specifically recognize HSP-CD36 complexes, isolated cells that express HSP-CD36 complexes, and isolated and recombinant cells that contain recombinant CD36 and HSP sequences. In addition, in various methods of the invention, sequences encoding CD36, an HSP, and CD36 are used for immunotherapy. Such compositions can be used, for example, in immunotherapy against proliferative disorders, infectious diseases, and other HSP-CD36-related disorders. Methods for the synthesis and production of such compositions are described herein.

5.1.1 Recombinant Expression

In various embodiments of the invention, sequences encoding CD36, an HSP, or other CD36 ligands are inserted into an expression vector for propagation and expression in recombinant cells. Thus, in one embodiment, CD36, HSP, or other CD36 ligand coding region is linked to a non-native promoter for expression in recombinant cells.

Based on the discovery by the Applicants, the extracellular portion of CD36 is likely responsible for recognizing and binding to HSPs and HSP-antigenic peptide complexes. HSP interaction with CD36 induces a signal transduction pathway which stimulates an immune response. Based on this invention, compositions comprising agonists and antagonists of CD36 and HSPs interactions can be used to modulate the immune response. Thus, recombinant CD36 polypeptides, complexes of CD36 and an HSP or HSP-antigenic peptide complexes, and recombinant cells expressing CD36 can be used in methods for immunotherapy and diagnostic methods described herein.

In various embodiments of the invention, sequences encoding CD36, and/or a heat shock protein, or fragments thereof, are inserted into an expression vector for propagation and expression in recombinant cells. An expression construct, as used herein, refers to a nucleotide sequence encoding a particular gene product, such as CD36 or HSP, operably associated with one or more regulatory regions which allows expression of the encoded gene product in an appropriate host cell. “Operably-associated” refers to an association in which the regulatory regions and the nucleotide sequence encoding the gene product to be expressed are joined and positioned in such a way as to permit transcription, and ultimately, translation.

The DNA may be obtained from known sequences derived from sequence databases by standard procedures known in the art by DNA amplification or molecular cloning directly from a tissue, cell culture, or cloned DNA (e.g., a DNA “library”). Any eukaryotic cell may serve as the nucleic acid source for obtaining the coding region of an hsp gene. Nucleic acid sequences encoding HSPs can be isolated from vertebrate, mammalian, as well as primate sources, including humans. Clones derived from genomic DNA may contain regulatory and intron DNA regions in addition to coding regions; clones derived from cDNA will contain only exon sequences. Whatever the source, the hsp gene should be cloned into a suitable vector for propagation of the gene.

Vectors based on E. coli are the most popular and versatile systems for high level expression of foreign proteins (Makrides, 1996, Microbiol Rev, 60:512-538). Non-limiting examples of regulatory regions that can be used for expression in E. coli may include but not limited to lac, trp, lpp, phoA, recA, tac, λP_{L,} and phage T3 and T7 promoters (Makrides, 1996, Microbiol Rev, 60:512-538). Non-limiting examples of prokaryotic expression vectors may include the λgt vector series such as λgt11 (Huynh et al., 1984 in “DNA Cloning Techniques”, Vol. I: A Practical Approach (D. Glover, ed.), pp. 49-78, IRL Press, Oxford), and the pET vector series (Studier et al., 1990, Methods Enzymol., 185:60-89). However, a potential drawback of a prokaryotic host-vector system is the inability to perform many of the post-translational processing events of mammalian cells. Thus, an eukaryotic host-vector system is preferred, a mammalian host-vector system is more preferred, and a human host-vector system is the most preferred.

The regulatory regions necessary for transcription of a CD36 sequence, for example, can be provided by the expression vector. A translation initiation codon (ATG) may also be provided to express a-nucleotide sequence encoding CD36 that lacks an initiation codon. In a compatible host-construct system, cellular proteins required for transcription, such as RNA polymerase and transcription factors, will bind to the regulatory regions on the expression construct to effect transcription of the CD36 sequence in the host organism. The precise nature of the regulatory regions needed for gene expression may vary from host cell to host cell. Generally, a promoter is required which is capable of binding RNA polymerase to initiate the transcription of an operably-associated nucleic acid sequence. Such regulatory regions may include those 5′-non-coding sequences involved with initiation of transcription and translation, such as the TATA box, the cap site, a CAAT box, and the like. The non-coding region 3′ to the coding sequence may contain transcriptional termination regulatory sequences, such as terminators and polyadenylation sites.

Both constitutive and inducible regulatory regions may be used for expression of CD36, HSP, or other CD36 ligand. It may be desirable to use inducible promoters when the conditions optimal for growth of the recombinant cells and the conditions for high level expression of the gene product are different. Examples of useful regulatory regions are provided in the next section below.

For expression of CD36, HSP, or other CD36 ligands gene product in mammalian host cells, a variety of regulatory regions can be used, for example, the SV40 early and late promoters, the cytomegalovirus (CMV) immediate early promoter, and the Rous sarcoma virus long terminal repeat (RSV-LTR) promoter. Inducible promoters that may be useful in mammalian cells include but are not limited to those associated with the metallothionein II gene, mouse mammary tumor virus glucocorticoid responsive long terminal repeats (MMTV-LTR), the β-interferon gene, and the Hsp70 gene (Williams et al., 1989, Cancer Res. 49:2735-42; Taylor et al., 1990, Mol. Cell Biol., 10:165-75). It may be advantageous to use heat shock promoters or stress promoters to drive expression of CD36 in recombinant host cells.

The following animal regulatory regions, which exhibit tissue specificity and have been utilized in transgenic animals, can also be used in tumor cells of a particular tissue type: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378).

The efficiency of expression of CD36 in a host cell may be enhanced by the inclusion of appropriate transcription enhancer elements in the expression vector, such as those found in SV40 virus, Hepatitis B virus, cytomegalovirus, immunoglobulin genes, metallothionein, β-actin (see Bittner et al., 1987, Methods in Enzymol. 153:516-544; Gorman, 1990, Curr. Op. in Biotechnol. 1:36-47).

The expression vector may also contain sequences that permit maintenance and replication of the vector in more than one type of host cell, or integration of the vector into the host chromosome. Such sequences may include but are not limited to replication origins, autonomously replicating sequences (ARS), centromere DNA, and telomere DNA. It may also be advantageous to use shuttle vectors that can be replicated and maintained in at least two types of host cells.

In addition, the expression vector may contain selectable or screenable marker genes for initially isolating or identifying host cells that contain DNA encoding CD36. For long term, high yield production of CD36, stable expression in mammalian cells is preferred. A number of selection systems may be used for mammalian cells, including, but not limited, to the herpes simplex virus thymidine kinase (Wigler et al., 1977, Cell 11:223), hypoxanthine-guanine phosphoribosyltransferase (Szybalski and Szybalski, 1962, Proc. Natl. Acad. Sci. USA 48:2026), and adenine phosphoribosyltransferase (Lowy et al., 1980, Cell 22:817) genes can be employed in tk⁻, hgprt⁻ or aprt⁻ cells, respectively. Also, antimetabolite resistance can be used as the basis of selection for dihydrofolate reductase (dhfr), which confers resistance to methotrexate (Wigler et al., 1980, Natl. Acad. Sci. USA 77:3567; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neomycin phosphotransferase (neo), which confers resistance to the aminoglycoside G-418 (Colberre-Garapin et al., 1981, J. Mol. Biol. 150:1); and hygromycin phosphotransferase (hyg), which confers resistance to hygromycin (Santerre et al., 1984, Gene 30:147). Other selectable markers, such as but not limited to histidinol and Zeocin™ can also be used.

In order to insert the DNA sequence encoding CD36, HSP, or other CD36 ligands into the cloning site of a vector, DNA sequences with regulatory functions, such as promoters, must be attached to DNA sequences encoding CD36, HSP, or other CD36 ligands, respectively. To do this, linkers or adapters providing the appropriate compatible restriction sites may be ligated to the ends of cDNA or synthetic DNA encoding CD36, by techniques well known in the art (Wu et al., 1987, Methods in Enzymol 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA by use of PCR with primers containing the desired restriction enzyme site.

In one embodiment, an expression construct comprising CD36 sequence operably associated with regulatory regions can be directly introduced into appropriate host cells for expression and production of CD36 without further cloning (see, for example, U.S. Pat. No. 5,580,859). The expression constructs may also contain DNA sequences that facilitate integration of CD36 sequence into the genome of the host cell, e.g., via homologous recombination. In this instance, it is not necessary to employ an expression vector comprising a replication origin suitable for appropriate host cells in order to propagate and express CD36 in the host cells.

Expression constructs containing cloned nucleotide sequence encoding CD36, an HSP, or other CD36 ligand, can be introduced into the host cell by a variety of techniques known in the art, including but not limited to, for prokaryotic cells, bacterial transformation (Hanahan, 1985, in DNA Cloning, A Practical Approach, 1 :109-136), and for eukaryotic cells, calcium phosphate mediated transfection (Wigler et al., 1977, Cell 11:223-232), liposome-mediated transfection (Schaefer-Ridder et al., 1982, Science 215:166-168), electroporation (Wolff et al., 1987, Proc Natl Acad Sci 84:3344), and microinjection (Cappechi, 1980, Cell 22:479-488).

For long term, high yield production of properly processed CD36, HSP, or other CD36 ligand, stable expression in mammalian cells is preferred. Cell lines that stably express CD36, HSP, or other CD36 ligand or CD36-peptide complexes may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while the desired gene product is expressed continuously.

The recombinant cells may be cultured under standard conditions of temperature, incubation time, optical density, and media composition. Alternatively, recombinant antigenic cells may be cultured under conditions emulating the nutritional and physiological requirements of the cancer cell or infected cell. However, conditions for growth of recombinant cells may be different from those for expression of CD36, HSPs, or other CD36 ligand, or antigenic peptide.

5.1.2 Peptide Synthesis

An alternative to producing peptides and polypeptides comprising HSP, CD36, or other CD36 ligand sequences, by recombinant techniques is peptide synthesis. For example, a peptide corresponding to a portion of an HSP or a CD36 peptide comprising the receptor-binding domain, which can be used as an antagonist in the therapeutic methods described herein, can be synthesized by use of a peptide synthesizer. Synthetic peptides corresponding to CD36 sequences useful for therapeutic methods described herein can also be produced synthetically. Conventional peptide synthesis may be used or other synthetic protocols well known in the art.

For example, peptides having the amino acid sequence of the CD36, an HSP, or other CD36 ligand, or an analog, mutein, fragment, or derivative thereof, may be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support i.e., polystyrene beads. The peptides are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc which is acid labile and Fmoc which is base labile. Details of appropriate chemistries, resins, protecting groups, protected amino acids and reagents are well known in the art and so are not discussed in detail herein (See, Atherton, et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting CD36, HSP, or other CD36 ligand peptides is accomplished using conventional procedures, such as preparative HPLC using gel permeation, partition and/or ion exchange chromatography. The choice of appropriate matrices and buffers are well known in the art and so are not described in detail herein.

In addition, analogs and derivatives of CD36, HSP, or other CD36 ligand proteins can be chemically synthesized. Furthermore, if desired, nonclassical amino acids or chemical amino acid analogs can be introduced as a substitution or addition into CD36, HSP, or other CD36 ligand sequence. Non-classical amino acids include but are not limited to the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

5.1.3 Antibodies Specific for CD36-HSP Complexes

Described herein are methods for the production of antibodies capable of specifically recognizing CD36 epitopes, HSP-CD36 complex epitopes or epitopes of conserved variants or peptide fragments of the receptor or receptor complexes. Such antibodies are useful for therapeutic and diagnostic methods of the invention.

Such antibodies may include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above. Such antibodies may be used, for example, in the detection of CD36 or HSP-CD36 complex in an biological sample. Such antibodies may also be utilized in conjunction with, for example, compound screening schemes, as described below, in Section 5.2, for the evaluation of the effect of test compounds on the interaction between HSPs and CD36.

Anti-CD36-HSP complex antibodies may additionally be used as a method for the inhibition of abnormal receptor product activity. Thus, such antibodies may, be utilized as part of treatment methods for HSP-CD36 related disorders, e.g., autoimmune disorders.

For the production of antibodies against CD36 or receptor complexes, various host animals may be immunized by injection with CD36 or HSP-CD36 complex, or a portion thereof. An antigenic portion of CD36 or HSP-CD36 complex can be readily predicted by algorithms known in the art.

Host animals may include, but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as CD36 or HSP-CD36 complex, or an antigenic functional derivative thereof. For the production of polyclonal antibodies, host animals such as those described above, may be immunized by injection with CD36 or HSP-CD36 complex, or portion thereof, supplemented with adjuvants as also described above.

Monoclonal antibodies, which are homogeneous populations of antibodies to a particular antigen, may be obtained by any technique that provides for the production of antibody molecules by continuous cell lines in culture. These include, but are not limited to, the hybridoma technique of Kohler and Milstein, (1975, Nature 256, 495-497; and U.S. Pat. No. 4,376,110), the human B-cell hybridoma technique (Kosbor et al., 1983, Immunology Today 4: 72; Cole et al., 1983, Proc. Natl. Acad. Sci. USA 80, 2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison, et al., 1984, Proc. Natl. Acad. Sci., 81: 6851-6855; Neuberger, et al., 1984, Nature 312: 604-608; Takeda, et al., 1985, Nature, 314: 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region (see, e.g., Cabilly et al., U.S. Pat. No. 4,816,567; and Boss et al., U.S. Pat. No. 4,816,397, which are incorporated herein by reference in their entirety).

In an additional embodiment of the invention, monoclonal antibodies can be produced in germ-free animals (see PCT International Publication No. WO 89/12690, published Dec. 12, 1989). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). Techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for CD36-HSP complex together with genes from a human antibody molecule of appropriate biological activity can also be used; such antibodies are within the scope of this invention.

Humanized antibodies are also provided (see U.S. Pat. No. 5,225,539 by Winter). An immunoglobuin light or heavy chain variable region consists of a “framework” region interrupted by three hypervariable regions, referred to as complementarity determining regions (CDRs). The extent of the framework region and CDRs have been precisely defined (see, “Sequences of Proteins of Immunological Interest”, Kabat, E. et al., U.S. Department of Health and Human Services (1983)). Briefly, humanized antibodies are antibody molecules from non-human species having one or more CDRs from the non-human species and a framework region from a human immunoglobulin molecule. Such CDRs-grafted antibodies have been successfully constructed against various antigens, for example, antibodies against IL-2 receptor as described in Queen et al., 1989, Proc. Natl. Acad. Sci. USA 86:10029; antibodies against the cell surface receptor CAMPATH as described in Riechmann et al., 1988, Nature 332:323; antibodies against hepatitis B in Co et al., 1991, Proc. Natl. Acad. Sci. USA 88:2869; as well as against viral antigens of the respiratory syncytial virus in Tempest et al., 1991, Bio-Technology 9:267. Humanized antibodies are most preferred for therapeutic use in humans.

Alternatively, techniques described for the production of single chain antibodies (U.S. Pat. No. 4,946,778; Bird, 1988, Science 242: 423-426; Huston et al., 1988, Proc. Natl. Acad. Sci. USA 85: 5879-5883; and Ward et al., 1989, Nature 334: 544-546) can be adapted to produce single chain antibodies against CD36 or HSP-CD36 complexes, or portions thereof Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include but are not limited to: the F(ab′)₂ fragments, which can be produced by pepsin digestion of the antibody molecule and the Fab fragments, which can be generated by reducing the disulfide bridges of the F(ab′)₂ fragments. Alternatively, Fab expression libraries may be constructed (Huse et al., 1989, Science, 246: 1275-1281) to allow rapid and easy identification of monoclonal Fab fragments with the desired specificity.

Antibodies to CD36 can, in turn, be utilized to generate anti-idiotype antibodies that “mimic” CD36, using techniques well known to those skilled in the art (see, e.g., Greenspan & Bona, 1993, FASEB J 7(5):437-444; and Nissinoff, 1991, J. Immunol. 147(8):2429-2438). For example antibodies which bind to CD36 ECD and competitively inhibit the binding of HSPs to CD36 can be used to generate anti-idiotypes that “mimic” the ECD and, therefore, bind and neutralize HSPs. Such neutralizing anti-idiotypes or Fab fragments of such anti-idiotypes can be used in therapeutic regimens to neutralize the native ligand and treat HSP-CD36-related disorders, such as immunological disorders, proliferative disorders, and infectious diseases.

Alternatively, antibodies to CD36 that can act as agonists of CD36 activity can be generated. Such antibodies will bind to CD36 and activate the signal transducing activity of the receptor. In addition, antibodies that act as antagonist of CD36 activity, i.e. inhibit the activation of CD36 would be particularly useful for treating autoimmune disorders, proliferative disorders, such as cancer, and infectious diseases. Methods for assaying for such agonists and antagonists are described in detail in Section 5.2, below.

5.2 Assays for the Identification of Compounds that Interact with CD36

The present invention is based on the discovery that CD36 recognizes HSP-antigenic peptide complexes and induces a signal transduction pathway which elicits an immune response. Thus, methods for identifying compounds that interact with the receptor, or enhance or block the function of the receptor, are included in the invention. The present invention provides in vitro and in vivo assay systems, described in the subsections below, which can be used to identify compounds or compositions that interact with CD36, or modulate the activity of CD36 and its interaction with HSPs or HSP-peptide complexes.

The invention provides screening methodologies useful in the identification of small molecules, proteins and other compounds which interact with CD36, or modulate the interaction of HSPs with CD36. Such compounds may bind CD36 genes or gene products with differing affinities, and may serve as regulators of receptor activity in vivo with useful therapeutic applications in modulating the immune response. For example, certain compounds that inhibit receptor function may be used in patients to downregulate destructive immune responses which are caused by cellular release of HSPs.

Methods to screen potential agents for their ability to interact with CD36, or modulate CD36 expression and activity can be designed based on the Inventors discovery of the receptor and its role in HSP or HSP-peptide complex binding and recognition. CD36 protein, nucleic acids, and derivatives can be used in screening assays to detect molecules that specifically bind to HSP proteins, derivatives, or nucleic acids, and thus have potential use as agonists or antagonists of CD36, to modulate the immune response. In a preferred embodiment, such assays are performed to screen for molecules with potential utility as anti-autoimmune disease, anti-cancer and anti-infective drugs (such as anti-viral drugs and antibiotic drugs), or lead compounds for drug development. For example, recombinant cells expressing CD36 nucleic acids can be used to recombinantly produce CD36 in these assays, to screen for molecules that interfere with the binding of HSPs to CD36. Similar methods can be used to screen for molecules that bind to CD36 derivatives or nucleic acids. Methods that can be used to carry out the foregoing are commonly known in the art.

Compounds capable of specifically binding to CD36 can be useful for immunotherapy. In one embodiment, an assay is disclosed for identifying compounds that specifically bind to CD36 comprising: (a) contacting CD36 with one or more test compounds under conditions conducive to binding; and (b) identifying one or more test compounds which specifically bind to CD36, such that a compound capable of specifically binding to CD36 is identified as a compound useful for immunotherapy.

Another method encompassed by the invention for identifying a compound useful for immunotherapy involves identifying a compound which modulates the binding of CD36 ligand to CD36. The term “CD36 ligand” as used herein, refers to a CD36 molecule capable of binding to CD36. Such CD36 ligands include, but are not limited to, lipoprotein complexes, thrombospondin 1, P. falciparum erythrocyte membrane protein 1 (PfEMP1), LDL, and phospholipids. The method comprises the steps of: (a) contacting CD36 with a CD36 ligand, or fragment, or analog, derivative or mimetic thereof, in the presence of one or more test compound; and (b) measuring the amount of CD36 ligand, or fragment, analog, derivative or mimetic thereof, bound to CD36, such that if the amount of bound CD36 ligand measured in (b) differs from the amount of bound CD36 measured in the absence of the test compound, then a compound useful for immunotherapy that modulates the binding of CD36 ligand to CD36 is identified.

In another embodiment, a method for identifying a compound useful for immunotherapy which modulates the interaction between CD36 and CD36 ligand is provided by the invention. This method comprises the steps of: (a) contacting CD36 with one or more test compounds; and (b) measuring the level of CD36 activity or expression, such that if the level of activity or expression measured in (b) differs from the level of CD36 activity in the absence of one or more test compounds, then a compound that modulates the interaction between CD36 and a CD36 ligand is identified.

In another embodiment, an assay for identifying a compound that modulates an HSP-CD36 mediated process is disclosed. This assay comprises: (a) contacting a test compound with an HSP and CD36; and (b) measuring the level CD36 activity or expression, such that if the level of activity or expression measured in (b) differs from the level of CD36 activity in the absence of the test compound, then a compound that modulates an HSP-CD36 mediated process is identified. In another embodiment, in which the compound identified is an antagonist which interferes with the interaction of the HSP with CD36, the method further comprises the step of determining whether the level interferes with the interaction of the HSP and CD36.

In another embodiment, a cell-based method for identifying a compound that modulates an HSP-CD36 mediated process is described. This method comprises the following steps: (a) contacting a test compound with a heat shock protein and a CD36-expressing cell; and (b) measuring the level of CD36 activity or expression in the cell, such that if the level of activity or expression measured in (b) differs from the level of CD36 activity in the absence of the test compound, then a compound that modulates an HSP-CD36 mediated process is identified.

In another embodiment, a receptor-ligand binding assay for identifying a compound that interacts with CD36, or modulates the binding of an HSP to CD36. One such method comprises: (a) contacting an HSP with CD36, or fragment, or analog, derivative or mimetic thereof, in the presence of a test compound; and (b) measuring the amount of heat shock protein bound to CD36, or fragment, analog, derivative or mimetic thereof, such that if the amount of bound heat shock protein measured in (b) differs from the amount of bound heat shock protein measured in the absence of the test compound, then a compound that modulates the binding of an HSP to CD36 is identified.

In another embodiment, a method for identifying a compound that modulates signal transduction by CD36-expressing cells is provided by the invention. In one embodiment, such a method comprises: (a) adding one or more test compounds to a mixture of CD36-expressing cells and a complex comprising a CD36 ligand and an antigenic molecule, under conditions conducive to CD36-mediated signal transduction stimulation; (b) measuring the level of signal tranducing activity by CD36 expressing cells, such that if the level measured in (b) differs from the level of said stimulation in the absence of the one or more test compounds, then a compound that modulates heat shock protein-mediated signal transduction stimulation by CD36-expressing cells is identified. In another embodiment, a test compound is added to a mixture of CD36-expressing cells and a complex consisting essentially of an HSP noncovalently associated with an antigenic molecule, under conditions conducive to CD36-mediated signal transducing stimulation; and the level of signal tranducing stimulation by the CD36-expressing cells is measured, such that if the level measured differs from the level of said stimulation in the absence of the test compound, then a compound that modulates HSP-mediated signal transducing stimulation by CD36-expressing cells is identified.

The assays of the present invention may be first optimized on a small scale (i.e., in test tubes), and then scaled up for high-throughput assays. In various embodiments, the in vitro screening assays of the present invention may be performed using purified components or cell lysates. In other embodiments, the screening assays may be carried out in intact cells in culture and in animal models. In accordance with the present invention, test compounds which are shown to modulate the activity of CD36 as described herein in vitro, will further be assayed in vivo, including cultured cells and animal models to determine if the test compound has the similar effects in vivo and to determine the effects of the test compound on cytokine release, intracellular Ca⁺⁺ release, T-cell cytotoxicity, tumor progression, nitric oxide release, chemokine release, the accumulation or degradation of positive and negative regulators, cellular proliferation, etc.

5.2.1 CD36-Ligand Binding Assays

The screening assays, described herein, can be used to identify compounds and compositions, including peptides and organic, non-protein molecules that interact with CD36, or that modulate the interaction between HSPs and CD36. Recombinant, synthetic, and otherwise exogenous compounds may have binding capacity and, therefore, may be candidates for pharmaceutical agents. Alternatively, the proteins and compounds include endogenous cellular components which interact with the identified genes and proteins in vivo. Such endogenous components may provide new targets for pharmaceutical and therapeutic interventions.

Thus, in a preferred embodiment, both naturally occurring and/or synthetic compounds (e.g., libraries of small molecules or peptides), may be screened for interacting with CD36 and/or modulating CD36 activity. In another series of embodiments, cell lysates or tissue homogenates may be screened for proteins or other compounds which bind to one of the normal or mutant genes and polypeptides.

The screening assays described herein may be used to identify small molecules, peptides or proteins, or derivatives, analogs and fragments thereof, that interact with CD36 and/or modulate the interaction of HSPs with CD36. Such compounds may be used as agonists or antagonists of the binding of CD36 ligands, such as HSPs and HSP complexes, by the cell surface receptor. For example, compounds that modulate CD36-ligand interaction include, but are not limited to, compounds that bind to CD36, thereby either inhibiting (antagonists) or enhancing (agonists) the binding of ligands, such as HSPs and HSP complexes, to the receptor, as well as compounds that bind to the ligand, such as for example, HSPs, thereby preventing or enhancing binding of ligand to the receptor. Compounds that affect gene activity (by affecting gene expression, including molecules, e.g., proteins or small organic molecules, that affect transcription or interfere with splicing events so that expression of the full length or truncated forms of CD36 can be modulated) can also be identified in the screens of the invention. Further, it should be noted that the assays described can also identify compounds that modulate CD36 ligand, for example HSP, signal transduction by CD36 (e.g., compounds which affect downstream signaling in CD36 signal transduction pathway). The identification and use of such compounds which affect signaling events downstream of CD36 and thus modulate effects of the receptor on the immune response are within the scope of the invention.

Compounds that affect CD36 gene activity (by affecting CD36 gene expression, including molecules, e.g., proteins or small organic molecules, that affect transcription or interfere with splicing events so that expression of the full length or the truncated form of CD36 can be modulated) can also be identified in the screens of the invention. However, it should be noted that the assays described can also identify compounds that modulate CD36 signal transduction (e.g., compounds which affect downstream signaling events which is activated by ligand binding to CD36). The identification and use of such compounds which affect signaling events downstream of CD36 and thus modulate effects of CD36 on the allergenic response are within the scope of the invention.

The screening assays described herein are designed to detect compounds that modulate, i.e. interfere with or enhance, ligand-receptor interactions, including HSP-CD36 interactions. As described in detail below, such assays are functional assays, such as binding assays, that can be adapted to a high-throughput screening methodologies.

Binding assays can be used to identify compounds that modulate the interaction between ligands, for example, HSPs, and CD36. In one aspect of the invention the screens may be designed to identify compounds that disrupt the interaction between CD36 and a ligand, such as, for example, HSPs or peptides derived from an HSP or another CD36 ligand. Such compounds will be useful as lead compounds for antagonists of HSP-CD36 related disorders and conditions, such as immune disorders, proliferative disorders, and infectious diseases.

Binding assays may be performed either as direct binding assays or as competition binding assays. In a direct binding assay, a test compound is tested for binding either to CD36 or to a CD36 ligand, such as an HSP. Then, in a second step, the test compound is tested for its ability to modulate the ligand-CD36 interaction. Competition binding assays, on the other hand, assess the ability of a test compound to compete with a ligand, i.e. an HSP, for binding to CD36.

In a direct binding assay, either the ligand and/or CD36 is contacted with a test compound under conditions that allow binding of the test compound to the ligand or the receptor. The binding may take place in solution or on a solid surface. Preferably, the test compound is previously labeled for detection. Any detectable compound may be used for labeling, such as but not limited to, a luminescent, fluorescent, or radioactive isotope or group containing same, or a nonisotopic label, such as an enzyme or dye. After a period of incubation sufficient for binding to take place, the reaction is exposed to conditions and manipulations that remove excess or non-specifically bound test compound. Typically, it involves washing with an appropriate buffer. Finally, the presence of a ligand-test compound (e.g., HSP-test compound) or a CD36-test compound complex is detected.

In a competition binding assay, test compounds are assayed for their ability to disrupt or enhance the binding of the ligand (e.g., HSP) to CD36. Labeled ligand (e.g., HSP) may be mixed with CD36 or fragment or derivative thereof, and placed under conditions in which the interaction between them would normally occur, with and without the addition of the test compound. The amount of labeled ligand (e.g., HSP) that binds CD36 may be compared to the amount bound in the presence or absence of test compound.

In a preferred embodiment, to facilitate complex formation and detection, the binding assay is carried out with one or more components immoblilized on a solid surface. In various embodiments, the solid support could be, but is not restricted to, polycarbonate, polystyrene, polypropylene, polyethlene, glass, nitrocellulose, dextran, nylon, polyacrylamide and agarose. The support configuration can include beads, membranes, microparticles, the interior surface of a reaction vessel such as a microtiter plate, test tube or other reaction vessel. The immobilization of CD36, or other component, can be achieved through covalent or non-covalent attachments. In one embodiment, the attachment may be indirect, i.e. through an attached antibody. In another embodiment, CD36 and negative controls are tagged with an epitope, such as glutathione S-transferase (GST) so that the attachment to the solid surface can be mediated by a commercially available antibody such as anti-GST (Santa Cruz Biotechnology).

For example, such an affinity binding assay may be performed using CD36 which is immobilized to a solid support. Typically, the non-mobilized component of the binding reaction, in this case either ligand (e.g., HSP) or the test compound, is labeled to enable detection. A variety of labeling methods are available and may be used, such as luminescent, chromophore, fluorescent, or radioactive isotope or group containing same, and nonisotopic labels, such as enzymes or dyes. In a preferred embodiment, the test compound is labeled with a fluorophore such as fluorescein isothiocyanate (FITC, available from Sigma Chemicals, St. Louis).

The labeled test compounds, or ligand (e.g., HSP) plus test compounds, are then allowed to contact with the solid support, under conditions that allow specific binding to occur. After the binding reaction has taken place, unbound and non-specifically bound test compounds are separated by means of washing the surface. Attachment of the binding partner to the solid phase can be accomplished in various ways known to those skilled in the art, including but not limited to chemical cross-linking, non-specific adhesion to a plastic surface, interaction with an antibody attached to the solid phase, interaction between a ligand attached to the binding partner (such as biotin) and a ligand-binding protein (such as avidin or streptavidin) attached to the solid phase, and so on.

Finally, the label remaining on the solid surface may be detected by any detection method known in the art. For example, if the test compound is labeled with a fluorophore, a fluorimeter may be used to detect complexes.

Preferably, CD36 is added to binding assays in the form of intact cells that express CD36, or isolated membranes containing CD36. Thus, direct binding to CD36 or the ability of a test compound to modulate a ligand-CD36complex (e.g., HSP-CD36 complex) may be assayed in intact cells in culture or in animal models in the presence and absence of the test compound. A labeled ligand (e.g., HSP) may be mixed with cells that express CD36, or to crude extracts obtained from such cells, and the test compound may be added. Isolated membranes may be used to identify compounds that interact with CD36. For example, in a typical experiment using isolated membranes, cells may be genetically engineered to express CD36. Membranes can be harvested by standard techniques and used in an in vitro binding assay. Labeled ligand (e.g., ¹²⁵I-labeled HSP) is bound to the membranes and assayed for specific activity; specific binding is determined by comparison with binding assays performed in the presence of excess unlabeled (cold) ligand. Alternatively, soluble CD36 may be recombinantly expressed and utilized in non-cell based assays to identify compounds that bind to CD36. The recombinantly expressed CD36 polypeptides or fusion proteins containing the extracellular domain (ECD) of CD36, or one or more subdomains thereof, can be used in the non-cell based screening assays. Alternatively, peptides corresponding to one or more of the CDs of CD36, or fusion proteins containing one or more of the CDs of CD36 can be used in non-cell based assay systems to identify compounds that bind to the cytoplasmic portion of CD36; such compounds may be useful to modulate the signal transduction pathway of CD36. In non-cell based assays the recombinantly expressed CD36 is attached to a solid substrate such as a test tube, microtiter well or a column, by means well known to those in the art (see Ausubel et al., supra). The test compounds are then assayed for their ability to bind to CD36.

Alternatively, the binding reaction may be carried out in solution. In this assay, the labeled component is allowed to interact with its binding partner(s) in solution. If the size differences between the labeled component and its binding partner(s) permit such a separation, the separation can be achieved by passing the products of the. binding reaction through an ultrafilter whose pores allow passage of unbound labeled component but not of its binding partner(s) or of labeled component bound to its partner(s). Separation can also be achieved using any reagent capable of capturing a binding partner of the labeled component from solution, such as an antibody against the binding partner, a ligand-binding protein which can interact with a ligand previously attached to the binding partner, and so on.

In one embodiment, for example, a phage library can be screened by passing phage from a continuous phage display library through a column containing purified CD36, or derivative, analog, fragment, or domain, thereof, linked to a solid phase, such as plastic beads. By altering the stringency of the washing buffer, it is possible to enrich for phage that express peptides with high affinity for CD36. Phage isolated from the column can be cloned and the affinities of the short peptides can be measured directly. Sequences for more than one oligonucleotide can be combined to test for even higher affinity binding to CD36. Knowing which amino acid sequences confer the strongest binding to CD36, computer models can be used to identify the molecular contacts between CD36 and the test compound. This will allow the design of non-protein compounds which mimic those contacts. Such a compound may have the same activity of the peptide and can be used therapeutically, having the advantage of being efficient and less costly to produce.

In another specific embodiment of this aspect of the invention, the solid support is membranes containing CD36 attached to a microtiter dish. Test compounds, for example, cells that express library members are cultivated under conditions that allow expression of the library members in the microtiter dish. Library members that bind to the protein (or nucleic acid or derivative) are harvested. Such methods, are described by way of example in Parmley and Smith, 1988, Gene 73:305-318; Fowlkes et al., 1992, BioTechniques 13:422-427; PCT Publication No. WO 94/18318; and in references cited hereinabove.

In another embodiment of the present invention, interactions between CD36 or ligand (e.g., HSP) and a test compound may be assayed in vitro. Known or unknown molecules are assayed for specific binding to CD36 nucleic acids, proteins, or derivatives under conditions conducive to binding, and then molecules that specifically bind to CD36 are identified. The two components can be measured in a variety of ways. One approach is to label one of the components with an easily detectable label, place it together with a test component(s) under conditions that allow binding to occur, perform a separation step which separates bound labeled component from unbound labeled component, and then measure the amount of bound component. In one embodiment, CD36 can be labeled and added to a test agent, using conditions that allow binding to occur. Binding of the test agent can be determined using polyacrylamide gel analysis to compare complexes formed in the presence and absence of the test agent.

In yet another embodiment, binding of ligand (e.g., HSP) to CD36 may be assayed in intact cells in animal models. A labeled ligand (e.g., HSP) may be administered directly to an animal, with and without a test compound. Signal transduction stimulation of the ligand (e.g., HSP) may be measured in the presence and the absence of test compound. For these assays, host cells to which the test compound is added may be genetically engineered to express CD36 and/or ligand (e.g., HSP), which may be transient, induced or constitutive, or stable. For the purposes of the screening methods of the present invention, a wide variety of host cells may be used including, but not limited to, tissue culture cells, mammalian cells, yeast cells, and bacteria. Mammalian cells such as macrophages or other cells that express CD36, i.e., cells of the monocytic lineage, liver parenchymal cells, fibroblasts, keratinocytes, neuronal cells, and placental syncytiotrophoblasts, may be a preferred cell type in which to carry out the assays of the present invention. Bacteria and yeast are relatively easy to cultivate but process proteins differently than mammalian cells.

5.2.2 Activity Assays

After identification of a test compound that interacts with, or modulates the interaction of a ligand (e.g., HSP) with CD36, the test compound can be further characterized to measure its effect on CD36 activity and the ligand-CD36 cellular signaling pathway. For example, the test compound may be characterized by testing its effect on ligand (e.g., HSP) CD36 cellular activity in vivo. Such assays include downstream signaling assays, assays for antigen-specific activation of cytotoxic T cells, nitric oxide assays, chemokine assay, and the like.

In various embodiments, a candidate compound identified in a primary assay may be tested for its effect on innate CD36 signaling transduction activity. For example, downstream signaling effects of activation which can be assayed include, but are not limited to: enhanced locomotion and chemotaxis of macrophages (Forrester et al., 1983, Immunology 50: 251-259), down regulation of proteinase synthesis, and elevation of intracellular calcium, inositol phosphates and cyclic AMP (Misra et al., 1993, Biochem. J., 290:885-891). Other innate immune responses that can be tested are release of cytokines (i.e., IL-12, IL1β, GMCSF, and TNFα), the release of nitric oxide, and the release of chemokine. Thus, as secondary assays, any identified candidate compound can be tested for changes in such activities in the presence and absence.

For example, in one embodiment, a chemotaxis assay can be used to further characterize a candidate identified by a primary screening assay. A number of techniques can be used to test chemotactic migration in vitro (see, e.g., Leonard et al., 1995, “Measurement of α and β Chemokines”, in Current Protocols in Immunology, 6.12.1-6.12.28, Ed. Coligan et al., John Wiley & Sons, Inc. 1995). For example, in one embodiment, a candidate compound can be tested for its ability to modulate the ability of CD36 to induce migration of cells that express the receptor using a chemokine gradient in a multiwell Boyden chemotaxis chamber. In a specific example of this method, a serial dilution of a ligand (e.g., an HSP)/CD36 antagonist or agonist test compound identified in the primary screen is placed in the bottom wells of the Boyden chemotaxis chamber. A constant amount of ligand is also added to the dilution series. As a control, at least one aliquot contains only ligand (e.g., HSP). The contribution of the antagonist or agonist compound to the chemotactic activity of CD36 is measured by comparing number of migrating cells on the lower surface of the membrane filter of the aliquots containing only ligand (e.g., HSP), with the number of cells in aliquots containing test compound and ligand (e.g., HSP). If addition of the test compound to the ligand (e.g., HSP) solution results in a decrease in the number of cells detected the membrane relative to the number of cells detected using a solution containing only ligand (e.g., HSP), then an antagonist of ligand (e.g., HSP) induction of chemotactic activity of CD36-expressing cells is identified.

In another embodiment, calcium flux assays can be used as secondary screens to further characterize modulators of ligand-CD36 interactions. Intracellular calcium ion concentration can be measured in cells that express CD36 in the presence of the ligand, in the presence and the absence of a test compound. For example, calcium mobilization can be detected and measured by flow cytometry, by labeling with fluorescent dyes that are trapped intracellularly. A fluorescent dye such as Indo-1 exhibits a change in emission spectrum upon binding calcium, the ratio of fluorescence produced by the calcium-bound dye to that produced by the unbound dye may be used to estimate the intracellular calcium concentration. In a specific embodiment, cells are incubated in a cuvette in media containing Indo-1 at 37° C. and are excited, and fluorescence is measured using a fluorimeter (Photon Technology Corporation, International). The ligand is added at a specific time point, in the presence and the absence of a test compound, EGTA is added to the cuvette to release and chelate total calcium, and the response is measured. Binding of ligand results in increased intracellular Ca²⁺ concentration in cells that express CD36. An agonist results in a relative increased intracellular Ca²⁺ concentration, whereas an antagonist results in a relative decreased intracellular Ca²⁺ concentration

Elevation of nitric oxide is also an indicator of CD36 activation. Thus, in another embodiment, nitric oxide assays can be used as screens to characterize modulators of ligand-CD36 interactions. Nitric oxide concentration can be measured in cells that express CD36 in the presence of the ligand, or in the presence and absence of a test compound. For example, after incubation for 20 hours, supernatant can be harvested and reacted with Greiss reagent (Nims, R. W., et al., 1996. Methods in Enzymology 268, 93). Such a reaction can be quantified by spectrophotometry.

Elevation of MCP-1 chemokine is also an indicator of CD36 activation. Thus, in another embodiment, MCP-1 chemokine assay can be used as screens to characterize modulators of ligand-CD36 interactions. MCP-1 chemokine concentration can be measured in cells that express CD36 in the presence of the ligand, or in the presence and absence of a test compound. For example, after incubation for 20 hours, supernatant can be harvested and analyzed by ELISA (enzyme linked immunosorbent assay) using an antibody specific for MCP-1 chemokine.

5.2.3 Compounds that Can be Screened in Accordance with the Invention

The screening assays described herein may be used to identify small molecules, peptides or proteins, or derivatives, analogs and fragments thereof, that interact with, or modulate the interaction of a ligand (e.g., HSP) with CD36. The compounds which may be screened in accordance with the invention include, but are not limited to small molecules, peptides, antibodies and fragments thereof, and other organic compounds (e.g., peptidomimetics) that bind to the ECD of CD36 and either inhibit the activity triggered by the natural ligand (i.e., antagonists) or mimic the activity triggered by the natural ligand (i.e., agonists), as well as small molecules, peptides, antibodies or fragments thereof, and other organic compounds. In one embodiment, such compounds include sequences of CD36, such as the ECD of CD36 (or a portion thereof), which can bind to and “neutralize” natural ligands, such as HSPs, LDL, etc. In another embodiment, such compounds include ligand sequences, such as HSP sequences which can bind to the active site of CD36, and block its activity.

Compounds that may be used for screening include, but are not limited to, peptides such as, for example, soluble peptides, including but not limited to members of random peptide libraries; (see, e.g., Lam et al., 1991, Nature 354:82-84; Houghten et al., 1991, Nature 354:84-86), and combinatorial chemistry-derived molecular library made of D- and/or L-configuration amino acids, phosphopeptides (including, but not limited to, members of random or partially degenerate, directed phosphopeptide libraries; see, e.g., Songyang et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

In one embodiment of the present invention, peptide libraries may be used as a source of test compounds that can be used to screen for modulators of CD36 interactions, such as HSP-CD36. Diversity libraries, such as random or combinatorial peptide or nonpeptide libraries can be screened for molecules that specifically bind to CD36. Many libraries are known in the art that can be used, e.g., chemically synthesized libraries, recombinant (e.g., phage display libraries), and in vitro translation-based libraries.

Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37(9):1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383.

Examples of phage display libraries are described in Scott & Smith, 1990, Science 249:386-390; Devlin et al., 1990, Science, 249:404-406; Christian et al., 1992, J. Mol. Biol. 227:711-718; Lenstra, 1992, J. Immunol. Meth. 152:149-157; Kay et al., 1993, Gene 128:59-65; and PCT Publication No. WO 94/18318 dated Aug. 18, 1994.

By way of examples of nonpeptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

Screening the libraries can be accomplished by any of a variety of commonly known methods. See, e.g., the following references, which disclose screening of peptide libraries: Parmley & Smith, 1989, Adv. Exp. Med. Biol. 251:215-218; Scott & Smith, 1990, Science 249:386-390; Fowlkes et al., 1992; BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar & Pabo, 1993, Science 263:671-673; and PCT Publication No. WO 94/18318.

In another embodiment of the present invention, the screening may be performed by adding the labeled ligand (e.g., HSP) to in vitro translation systems such as a rabbit reticulocyte lysate (RRL) system and then proceeding with in vitro priming reaction. In vitro translation-based libraries include but are not limited to those described in PCT Publication No. WO 91/05058 dated Apr. 18, 1991; and Mattheakis et al., 1994, Proc. Natl. Acad. Sci. USA 91:9022-9026.

Compounds that can be tested and identified methods described herein can include, but are not limited to, compounds obtained from any commercial source, including Aldrich (Milwaukee, Wis. 53233), Sigma Chemical (St. Louis, Mo.), Fluka Chemie AG (Buchs, Switzerland) Fluka Chemical Corp. (Ronkonkoma, N.Y.;), Eastman Chemical Company, Fine Chemicals (Kingsport, Tenn.), Boehringer Mannheim GmbH (Mannheim, Germany), Takasago (Rockleigh, N.J.), SST Corporation (Clifton, N.J.), Ferro (Zachary, La. 70791), Riedel-deHaen Aktiengesellschaft (Seelze, Germany), PPG Industries Inc., Fine Chemicals (Pittsburgh, Pa. 15272). Further any kind of natural products may be screened using the methods of the invention, including microbial, fungal, plant or animal extracts.

Furthermore, diversity libraries of test compounds, including small molecule test compounds, may be utilized. For example, libraries may be commercially obtained from Specs and BioSpecs B.V. (Rijswijk, The Netherlands), Chembridge Corporation (San Diego, Calif.), Contract Service Company (Dolgoprudny, Moscow Region, Russia), Comgenex USA Inc. (Princeton, N.J.), Maybridge Chemicals Ltd. (Cornwall PL34 OHW, United Kingdom), and Asinex (Moscow, Russia).

Still further, combinatorial library methods known in the art, can be utilize, including, but not limited to: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, 1997, Anticancer Drug Des.12:145). Combinatorial libraries of test compounds, including small molecule test compounds, can be utilized, and may, for example, be generated as disclosed in Eichler & Houghten, 1995, Mol. Med. Today 1:174-180; Dolle, 1997, Mol. Divers. 2:223-236; and Lam, 1997, Anticancer Drug Des. 12:145-167.

Examples of methods for the synthesis of molecular libraries can be found in the art, for example in: DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented in solution (e.g., Houghten, 1992, BioTechniques 13:412-421), or on beads (Lam, 1991, Nature 354:82-84), chips (Fodor, 1993, Nature 364:555-556), bacteria (U.S. Pat. No. 5,223,409), spores (U.S. Pat. Nos. 5,571,698; 5,403,484; and 5,223,409), plasmids (Cull et a., 1992, Proc. Natl. Acad. Sci. USA 89:1865-1869) or phage (Scott and Smith, 1990, Science 249:386-390; Devlin, 1990, Science 249:404-406; Cwirla et al., 1990, Proc. Natl. Acad. Sci. USA 87:6378-6382; and Felici, 1991, J. Mol. Biol. 222:301-310).

Screening the libraries can be accomplished by any of a variety of commonly known methods. See, e.g., the following references, which disclose screening of peptide libraries: Parmley & Smith, 1989, Adv. Exp. Med. Biol. 251:215-218; Scott & Smith, 1990, Science 249:386-390; Fowlkes et al., 1992; BioTechniques 13:422-427; Oldenburg et al., 1992, Proc. Natl. Acad. Sci. USA 89:5393-5397; Yu et al., 1994, Cell 76:933-945; Staudt et al., 1988, Science 241:577-580; Bock et al., 1992, Nature 355:564-566; Tuerk et a., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar & Pabo, 1993, Science 263:671-673; and PCT Publication No. WO 94/18318.

5.3 Identification of Fragments of CD36 and/or CD36 Ligands, such as HSPS, Useful for Immunotherapy

The invention also encompasses methods for identifying ligand-binding CD36 fragments (such as “HSP-binding domains”), and analogs, muteins, or derivatives thereof, which are capable of binding to CD36 ligand peptide, such as HSP peptide complexes. Such ligand-binding CD36 fragment, e.g., HSP-binding domains, can then be tested for activity in vivo and in vitro using CD36/ligand binding assays, described in Section 5.2.1, above. In one embodiment, such a method for identifying a CD36 fragment capable of binding a heat shock protein comprises the steps of: (a) contacting a heat shock protein with one or more CD36 fragments; and (b) identifying a CD36 polypeptide fragment which specifically binds to the heat shock protein.

Ligand-binding domains, e.g., HSP-binding domains, of CD36 capable of binding ligand-antigenic peptide complexes, such as HSP-antigenic peptide complexes, can be further tested for activity using either in vivo binding assays, or CTL assays, such as those described in Section 5.2.2, above. For example, one such method for identifying a CD36 fragment capable of inducing an HSP-CD36 mediated process comprises the steps of: (a) contacting a heat shock protein with a cell expressing a CD36 fragment; and (b) measuring the level of CD36 activity in the cell, such that if the level of the HSP-CD36 mediated process or activity measured in (b) is greater than the level of CD36 activity in the absence of the CD36 fragment, then a CD36 fragment capable of inducing an HSP-CD36 mediated process is identified. Depending on their behavior in such assays, such molecules can be used to either enhance or, alternatively, block the function of the receptor when administered or expressed in vivo. For example, these assays can be used to identify CD36 HSP-binding domains which can bind HSP-antigen complexes and negatively interfere with signal transducing activity. These antagonists could be used to downregulate immune responses which are caused by cellular release of HSPs. Alternatively, certain CD36 HSP-binding domains may be used to enhance HSP-antigen complex uptake and signaling. Such agonists could be administered or expressed in subjects to elicit an immune response against an antigen of interest.

In another embodiment, the invention encompasses methods for identifying a ligand fragment, such as HSP fragments, which are capable of binding CD36 (“CD36-binding domains”), and analogs, muteins, or derivatives thereof. As described for assays for CD36-related polypeptides described above, such CD36-binding domains can then be tested for activity in vivo and in vitro using the binding assays described in Section 5.2.1, above. For example, one such method for identifying a heat shock protein fragment capable of binding CD36 comprises: (a) contacting CD36 with one or more heat shock protein fragments; and (b) identifying a heat shock protein fragment which specifically binds to CD36.

Ligand fragments, such as HSP fragments, of interest may be further tested in cells, using in vivo binding assays, or CTL assays, such as those described in Section 5.2.2, above. For example, in one embodiment, such a method for identifying a heat shock protein fragment capable of inducing an HSP-CD36 mediated process comprises: a) contacting an fragment with a cell expressing a heat shock protein; and b) measuring the level of CD36 activity in the cell, such that if the level of the HSP-CD36 mediated process or activity measured in (b) is greater than the level of CD36 activity in the absence of said heat shock protein fragment, then an HSP fragment capable of inducing an HSP-CD36 mediated process is identified. Alternatively, CD36-binding domains could be used to block HSP uptake by CD36. In one embodiment, such HSP fragments comprising CD36-binding domain sequences could be used to construct recombinant fusion proteins, comprised of a heat shock protein CD36-binding domain and an antigenic peptide sequence. Such recombinant fusion proteins may be used to elicit an immune response and to treat or prevent immune diseases and disorders (Suzue et al., 1997, Proc. Natl. Acad. Sci. U.S.A. 94: 13146-51).

CD36 fragments, analogs, muteins, and derivatives and/or ligand (e.g., HSP) fragments, analogs, muteins, and derivatives of the invention may be produced by recombinant DNA techniques, synthetic methods, or by enzymatic or chemical cleavage of native CD36 and/or ligands (e.g., HSPs).

Any eukaryotic cell may serve as the nucleic acid source for obtaining the coding region of a CD36 ligand (e.g., HSP) gene. Nucleic acid sequences encoding ligand, e.g., HSPs, and or CD36 can be isolated from vertebrate, mammalian, as well as primate sources, including humans. Amino acid sequences and nucleotide sequences of naturally occurring ligands, e.g., HSPs, and CD36 are generally available in sequence databases, such as Genbank.

The DNA may be obtained by standard procedures known in the art by DNA amplification or molecular cloning directly from a tissue, cell culture, or cloned DNA (e.g., a DNA “library”). Clones derived from genomic DNA may contain regulatory and intron DNA regions in addition to coding regions; clones derived from cDNA will contain only exon sequences. In a preferred embodiment, DNA can be amplified from genomic or cDNA by polymerase chain reaction (PCR) amplification using primers designed from the known sequence of an ligand, e.g., HSP or other CD36 ligand. The polymerase chain reaction (PCR) is commonly used for obtaining genes or gene fragments of interest. For example, a nucleotide sequence encoding a fragment of any desired length can be generated using PCR primers that flank the nucleotide sequence encoding the peptide-binding domain. Alternatively, a CD36 ligand, e.g., HSP, or other CD36 ligand receptor gene sequence can be cleaved at appropriate sites with restriction endonuclease(s) if such sites are available, releasing a fragment of DNA encoding the peptide-binding domain. If convenient restriction sites are not available, they may be created in the appropriate positions by site-directed mutagenesis and/or DNA amplification methods known in the art (see, for example, Shankarappa et a., 1992, PCR Method Appl. 1:277-278). The DNA fragment that encodes a fragment of the ligand (e.g., HSP) or CD36 gene is then isolated, and ligated into an appropriate expression vector, care being taken to ensure that the proper translation reading frame is maintained. Alternatives to isolating the genomic DNA include, but are not limited to, chemically synthesizing the gene sequence itself from a known sequence or making cDNA to the mRNA which encodes the ligand (e.g., HSP) and/or CD36.

Any technique for mutagenesis known in the art can be used to modify individual nucleotides in a DNA sequence, for purpose of making amino acid substitution(s) in the expressed peptide sequence, or for creating/deleting restriction sites to facilitate further manipulations. Such techniques include but are not limited to, chemical mutagenesis, in vitro site-directed mutagenesis (Hutchinson, C., et al., 1978, J. Biol. Chem 253:6551), oligonucleotide-directed mutagenesis (Smith, 1985, Ann. Rev. Genet. 19:423-463; Hill et al., 1987, Methods Enzymol. 155:558-568), PCR-based overlap extension (Ho et al., 1989, Gene 77:51-59), PCR-based megaprimer mutagenesis (Sarkar et al., 1990, Biotechniques, 8:404-407), etc. Modifications can be confirmed by double stranded dideoxy DNA sequencing.

An alternative to producing CD36 and/or ligand (e.g., HSP) fragments by recombinant techniques is peptide synthesis. For example, a peptide corresponding to a portion of CD36 and/or ligand (e.g., HSP) comprising the substrate-binding domain, or which binds peptides in vitro, can be synthesized by use of a peptide synthesizer. Conventional peptide synthesis may be used or other synthetic protocols well known in the art.

In addition, analogs and derivatives of CD36 and/or ligand (e.g., HSP) can be chemically synthesized. Furthermore, if desired, nonclassical amino acids or chemical amino acid analogs can be introduced as a substitution or addition into CD36 and/or ligand (e.g., HSP) sequence. Non-classical amino acids include but are not limited to the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general.

CD36 and/or ligand (e.g., HSP) peptides, or a mutant or derivative thereof, may be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support i.e., polystyrene beads. The peptides are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc which is acid labile and Fmoc which is base labile. Details of appropriate chemistries, resins, protecting groups, protected amino acids and reagents are well known in the art and so are not discussed in detail herein (See, Atherton, et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting fragment is accomplished using conventional procedures, such as preparative HPLC using gel permeation, partition and/or ion exchange chromatography. The choice of appropriate matrices and buffers are well known in the art and so are not described in detail herein.

In an alternative embodiment, fragments of CD36 and/or ligand (e.g., HSP) may be obtained by chemical or enzymatic cleavage of native or recombinant CD36 and/or ligand (e.g., HSP) molecules. Specific chemical cleavage can be performed by cyanogen bromide, NaBH₄, acetylation, formylation, oxidation, reduction, metabolic synthesis in the presence of tunicamycin, etc. Endoproteases that cleave at specific sites can also be used. Such proteases are known in the art, including, but not limited to, trypsin, α-chymotrypsin, V8 protease, papain, and proteinase K (see Ausubel et al., (eds.), in “Current Protocols in Molecular Biology”, Greene Publishing Associates and Wiley Interscience, New York, 17.4.6-17.4.8). CD36 and/or ligand (e.g., HSP) amino acid sequence of interest can be examined for the recognition sites of these proteases. An enzyme is chosen which can release a peptide-binding domain or peptide-binding fragment. CD36 and/or ligand (e.g., HSP) molecule is then incubated with the protease, under conditions that allow digestion by the protease and release of the specifically designated peptide-binding fragments. Alternatively, such protease digestions can be carried out blindly, i.e., not knowing which digestion product will contain the peptide-binding domain, using specific or general specificity proteases, such as proteinase K or pronase.

Once a fragment is prepared, the digestion products may be purified as described above, and subsequently tested for the ability to bind peptide or for immunogenicity. Methods for determining the immunogenicity of ligand (e.g., HSP) complexes by cytotoxicity tests are described in Section 5.2.2.

5.4 Drug Design

Upon identification of a compound that interacts with CD36, or modulates the interaction of a CD36 ligand, such as an HSP, with CD36, such a compound can be further investigated to test for an ability to alter the immune response. In particular, for example, the compounds identified via the present methods can be further tested in vivo in accepted animal models of HSP-CD36-mediated processes and HSP-CD36 related disorders, such as, e.g., immune disorders, proliferative disorders, and infectious diseases.

Computer modeling and searching technologies permit identification of compounds, or the improvement of already identified compounds, which can modulate the interaction of CD36 with its ligand, e.g., an HSP. Having identified such a compound or composition, the active sites or regions are identified. Such active sites might typically be ligand binding sites. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, which may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential CD36-modulating compounds.

Alternatively, these methods can be used to identify improved modulating compounds from an already known modulating compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the active sites of either CD36 or the HSP, and other ligands and their analogs, will be apparent to those of skill in the art.

Examples of molecular modeling systems are the CHARMm and QUANTA programs (Polygen Corporation, Waltham, Mass.). CHARMm performs the energy minimization and molecular dynamics functions. QUANTA performs the construction, graphic modelling and analysis of molecular structure. QUANTA allows interactive construction, modification, visualization, and analysis of the behavior of molecules with each other.

A number of articles review computer modeling of drugs interactive with specific proteins, such as Rotivinen et al.) 1988, Acta Pharmaceutical Fennica 97:159-166); Ripka (1988 New Scientist 54-57); McKinaly and Rossmann (1989, Annu. Rev. Pharmacol. Toxiciol. 29:111-122); Perry and Davies, OSAR: Quantitative Structure-Activity Relationships in Drug Design pp. 189-193 Alan R. Liss, Inc. 1989; Lewis and Dean (1989, Proc. R. Soc. Lond. 236:125-140 and 141-162); and, with respect to a model receptor for nucleic acid components, Askew et al. (1989, J. Am. Chem. Soc. 111: 1082-1090). Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

5.5 Diagnostic Uses

CD36 was initially identified as a heat shock protein receptor due to its interaction with gp96, which is exclusively intracellular and is released as a result of necrotic but not apoptotic cell death. Thus, gp96 binding to CD36 may act as a sensor of necrotic cell death. As such, CD36-ligand complexes may be used to detect and diagnose proliferative disorders, such as cancer, autoimmune disorders and infectious disease. Therefore, CD36 proteins, analogues, derivatives, and subsequences thereof, CD36 nucleic acids (and sequences complementary thereto), and anti-CD36 antibodies, have uses in detecting and diagnosing such disorders.

CD36 and CD36 nucleic acids can be used in assays to detect, prognose, or diagnose immune system disorders that may result in tumorigenesis, carcinomas, adenomas etc, and viral disease.

The molecules of the present invention can be used in assays, such as immunoassays, to detect, prognose, diagnose, or monitor various conditions, diseases, and disorders affecting expression, or monitor the treatment thereof. In particular, such an immunoassay is carried out by a method comprising contacting a sample derived from a patient with an HSP-CD36 specific antibody under conditions such that immunospecific binding can occur, and detecting or measuring the amount of any immunospecific binding by the antibody. In a specific aspect, such binding of antibody, in tissue sections, can be used to detect aberrant localization or aberrant (e.g., low or absent) levels of CD36. In a specific embodiment, antibody to CD36 can be used to assay a patient tissue or serum sample for the presence of CD36 where an aberrant level of CD36 is an indication of a diseased condition. By “aberrant levels,” is meant increased or decreased levels relative to that present, or a standard level representing that present, in an analogous sample from a portion of the body or from a subject not having the disorder.

The immunoassays which can be used include but are not limited to competitive and non-competitive assay systems using techniques such as western blots, immunohisto-chemistry radioimmunoassays, ELISA, “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few.

CD36 genes and related nucleic acid sequences and subsequences, including complementary sequences, can also be used in hybridization assays. CD36 nucleic acid sequences, or subsequences thereof, comprising about at least 8 nucleotides, can be used as hybridization probes. Hybridization assays can be used to detect, prognose, diagnose, or monitor conditions, disorders, or disease states associated with aberrant changes in expression and/or activity as described supra. In particular, such a hybridization assay is carried out by a method comprising contacting a sample containing nucleic acid with a nucleic acid probe capable of hybridizing to CD36 DNA or RNA, under conditions such that hybridization can occur, and detecting or measuring any resulting hybridization.

In specific embodiments, diseases and disorders involving decreased immune responsiveness during an infection or malignant disorder can be diagnosed, or their suspected presence can be screened for, or a predisposition to develop such disorders can be detected, by detecting decreased levels of CD36 protein, CD36 RNA, or CD36 functional activity (e.g., binding to HSP, antibody-binding activity etc.), or by detecting mutations in CD36 RNA, DNA or CD36 protein (e.g., translocations in CD36 nucleic acids, truncations in CD36 gene or protein, changes in nucleotide or amino acid sequence relative to wild-type CD36) that cause decreased expression or activity of CD36. Such diseases and disorders include but are not limited to those described in Sections 5.7, 5.8, and 5.9. By way of example, levels of CD36 protein can be detected by immunoassay, levels of CD36 RNA can be detected by hybridization assays (e.g., Northern blots, in situ-hybridization), CD36 activity can be assayed by measuring binding activities in vivo or in vitro. Translocations, deletions, and point mutations in CD36 nucleic acids can be detected by Southern blotting, FISH, RFLP analysis, SSCP, PCR using primers, preferably primers that generate a fragment spanning at least most of CD36 gene, sequencing of CD36 genomic DNA or cDNA obtained from the patient, etc.

In a preferred embodiment, levels of CD36 mRNA or protein in a patient sample are detected or measured relative to the levels present in an analogous sample from a subject not having the malignancy or hyperproliferative disorder. Decreased levels indicate that the subject may develop, or have a predisposition to developing, viral infection, malignancy, or hyperproliferative disorder.

In another specific embodiment, diseases and disorders involving a deficient immune responsiveness resulting in cell proliferation or in which cell proliferation is desirable for treatment, are diagnosed, or their suspected presence can be screened for, or a predisposition to develop such disorders can be detected, by detecting increased levels of CD36 protein, RNA, or CD36 functional activity (e.g., HSP binding or antibody, etc.), or by detecting mutations in CD36 RNA, DNA or protein (e.g., translocations in CD36 nucleic acids, truncations in the gene or protein, changes in nucleotide or amino acid sequence relative to wild-type ) that cause increased expression or activity of CD36. Such diseases and disorders include, but are not limited to, those described in Sections 5.7, 5.8, and 5.9. By way of example, levels of CD36 protein, levels of CD36 RNA, CD36 binding activity, and the presence of translocations or point mutations can be determined as described above.

In a specific embodiment, levels of CD36 mRNA or protein in a patient sample are detected or measured, relative to the levels present in an analogous sample from a subject not having the disorder, in which increased levels indicate that the subject has, or has a predisposition to, an autoimmune disorder.

Kits for diagnostic use are also provided, that comprise in one or more containers an anti-CD36 antibody, and, optionally, a labeled binding partner to the antibody. Alternatively, the anti-CD36 antibody can be labeled (with a detectable marker, e.g., a chemiluminescent, enzymatic, fluorescent, or radioactive moiety). A kit is also provided that comprises in one or more containers a nucleic acid probe capable of hybridizing to CD36 RNA. In a specific embodiment, a kit can comprise in one or more containers a pair of primers (e.g., each in the size range of 6-30 nucleotides) that are capable of priming amplification [e.g., by polymerase chain reaction (see e.g., Innis et al., 1990, PCR Protocols, Academic Press, Inc., San Diego, Calif.), ligase chain reaction (see EP 320,308) use of Qβ replicase, cyclic probe reaction, or other methods known in the art] under appropriate reaction conditions of at least a portion of an nucleic acid. A kit can optionally further comprise in a container a predetermined amount of a purified CD36 protein or nucleic acid, e.g., for use as a standard or control.

5.6 Therapeutic Uses

The invention further encompasses methods for modulating the immune response. CD36 recognizes HSP proteins such as gp96 and stimulate chemokine and nitric oxide release (e.g., HSP-antigenic peptide complexes) for the purpose of stimulating the immune system and eliciting an immune response. Thus, the compositions and methods of the invention may be used for therapeutic treatment of HSP-CD36 related disorders and conditions, such as autoimmune diseases, cancer and infectious diseases. In particular, as described in detail hereinbelow, recombinant cells comprising CD36 complexes, such as HSP-antigenic peptide complexes, antibodies and other compounds that interact with CD36, or modulate the interaction between CD36 and its ligands, e.g., HSP, as well as other compounds that modulate HSP-CD36-mediated processes may be used to elicit, or block, an immune response to treat such HSP-CD36 related disorders and conditions.

5.6.1 Therapeutic Use of Identified Agonists and Antagonists

Compounds, such as those identified by screening methods provided herein, that interact with CD36, or modulate the interaction between CD36 and its ligand, e.g., HSP, can be useful as therapeutics. Such compounds, include, but are not limited to, agonists, antagonists, such as antibodies, antisense RNAs and ribozymes. Compounds which interfere with ligand (e.g., HSP) CD36-interaction can be used to block an immune response, and can be used to treat autoimmune responses and conditions. Other antibodies, agonists, antagonists, antisense RNAs and ribozymes may upregulate ligand (e.g., HSP)-CD36 interaction, activity, or expression, and would enhance the uptake of antigen complexes (e.g., HSP-antigen complexes), and therefore be useful in stimulating the host's immune system prior to, or concurrent with, the administration of a vaccine. Described below are methods and compositions for the use of such compounds in the treatment of HSP-CD36 related disorders, such as immune disorders, proliferative disorders, and infectious diseases.

In one embodiment an antagonist of CD36-ligand (e.g., HSP-CD36) interaction is used to block the immune response. Such antagonists include compounds that interfere with binding of a ligand (e.g., an HSP) to the receptor by competing for binding to CD36, the ligand, or the ligand-CD36 complex.

In one embodiment, the antagonist is an antibody specific for CD36, or a fragment thereof which contains the HSP ligand binding site. In another embodiment the antagonist is an antibody specific for an HSP, which interferes with binding of the HSP to the receptor.

In another embodiment, the antagonist is a peptide which comprises at least contiguous 10 amino acids of an HSP sequence. Such a peptide can bind to the ligand binding site of CD36 a block the interaction of an HSP or HSP complex. In another embodiment, the antagonist is a peptide which comprises at least contiguous 10 amino acids of CD36 sequence, which, like an HSP, can bind to CD36 and interfere with the binding and signal transducing activity. In yet another embodiment, the antagonist is a peptide which comprises at least contiguous 10 amino acids of CD36 sequence, in particular the ECD of CD36 (or a portion thereof), which can bind to and “neutralize” natural ligands, such as HSPs, LDL, etc.

Such peptides may be produced synthetically or by using standard molecular biology techniques. Amino acid sequences and nucleotide sequences of naturally occurring CD36 ligands, such as HSPs are generally available in sequence databases, such as GenBank. Computer programs, such as Entrez, can be used to browse the database, and retrieve any amino acid sequence and genetic sequence data of interest by accession number. Methods for recombinant and synthetic production of such peptides are described in Sections 5.1.1 and 5.1.2.

Additionally, compounds, such as those identified via techniques such as those described hereinabove, in Section 5.2, that are capable of modulating CD36 gene product activity can be administered using standard techniques that are well known to those of skill in the art.

5.6.1.1 Competitive Antagonists of CD36-Ligand Interactions

In one embodiment an antagonist of CD36-ligand (e.g., HSP-CD36 ) interaction is used to block the immune response to an antigen complex, e.g., to treat an auto-immune disorder. Such antagonists include molecules that interfere with binding by binding to CD36, thereby interfering with binding of a ligand (e.g., HSP) to the receptor. An example of this type of competitive inhibitor is an antibody to CD36, or a fragment of CD36 which contains an HSP ligand binding site.

A CD36-ligand (e.g., HSP) competitive inhibitor can be any type of molecule, including but not limited to a protein, nucleic acid or drug. In a preferred embodiment, an HSP-CD36 competitive inhibitor is a CD36-binding or an HSP-binding peptide. Examples of such peptides are provided below.

5.6.1.1.1 HSP-Binding Peptides

CD36 Peptides

In one embodiment of the present invention, a HSP-CD36 competitive antagonist is a CD36 peptide, preferably a soluble peptide, that can bind to HSPs and therefore competitively inhibit HSP binding to the native receptor.

Functional expression of HSP-binding portions of CD36 is preferably carried out. Briefly, to maintain proper folding, the protein is expressed as a GST fusion, expressed recombinantly, the GST portion cleaved, uncleaved protein removed on GSH-Sepharose, and cleaved protein refolded. Since the complement repeats bind to calcium, proper folding is assayed by measuring the binding of the refolded protein to calcium.

In a specific mode of the embodiment, an HSP-binding portion of CD36 consists of or comprises at least one complement repeat. In another specific mode of the embodiment, an HSP-binding portion of CD36 comprises a cluster of complement repeats, most preferably C1-II. In other modes of the embodiment, the HSP-binding portion consists of at least 10, more preferably at least 20, yet more preferably at least 30, yet more preferably at least 40, and most preferably at least 80 (continuous) amino acids. In specific modes of the embodiment, such fragments are not larger than 40-45 amino acids. In other specific modes of the embodiment, such fragments are not larger than 80-90 amino acids.

Derivatives or analogs of HSP-binding portions of CD36 are also contemplated as competitive antagonists of HSP-CD36 complexes. Such derivative or analogs include but are not limited to those molecules comprising regions that are substantially homologous to the extracellular domain of CD36 or fragments thereof (e.g., in various embodiments, at least 60% or 70% or 80% or 90% or 95% identity over an amino acid sequence of identical size or when compared to an aligned sequence in which the alignment is done by a computer homology program known in the art) or whose encoding nucleic acid is capable of hybridizing to a sequence encoding a CD36 HSP-binding sequence, under stringent, moderately stringent, or nonstringent conditions. In certain specific embodiments, a CD36 derivative is a chimeric or fusion protein comprising an HSP-binding portion of CD36, preferably consisting of at least one complement repeat of C1-II joined at its amino- or carboxy-terminus via a peptide bond to an amino acid sequence of a different protein. Such a chimeric protein can be produced recombinantly as described above, by omitting the cleavage repurification steps.

Other HSP-binding CD36 derivatives can be made by altering CD36 coding sequences by substitutions, additions or deletions that provide for functionally equivalent molecules. Due to the degeneracy of nucleotide coding sequences, other DNA sequences which encode substantially the same amino acid sequence as CD36 gene or gene fragment may be used in the practice of the present invention. Selection of suitable alterations and production of HSP-binding CD36 derivatives can be made applying the same principles described above for CD36 derivatives and using the general methods described in Sections 5.1.1 and 5.1.2.

HSP Peptides

In another mode of the embodiment, the antagonist is a peptide which comprises at least contiguous 10 amino acids of an HSP sequence. Such a peptide can bind to the ligand binding site of CD36 and block the interaction of an HSP or HSP complex.

Such peptides may be produced synthetically or by using standard molecular biology techniques. Amino acid sequences and nucleotide sequences of naturally occurring HSPs are generally available in sequence databases, such as GenBank. Computer programs, such as Entrez, can be used to browse the database, and retrieve any amino acid sequence and genetic sequence data of interest by accession number. Methods for recombinant and synthetic production of such peptides are described in Sections 5.1.1 and 5.1.2.

Additionally, compounds, such as those identified via techniques such as those described hereinabove, in Section 5.2, that are capable of modulating gene product activity can be administered using standard techniques that are well known to those of skill in the art.

5.6.2 Therapeutic Use of CD36 against Cancer and Infectious Diseases

In another embodiment, symptoms of certain CD36 gene disorders, such as autoimmune disorders, or proliferative or differentiative disorders causing tumorigenesis or cancer, may be ameliorated by modulating the level of CD36 gene expression and/or CD36 gene product activity. In one embodiment, for example, a decrease in CD36 gene expression may be useful to decrease CD36 activity, and ameliorate the symptoms of an autoimmune disorder. In this case, the level of CD36 gene expression may be decreased by using CD36 gene sequences in conjunction with well-known antisense, gene “knock-out,” ribozyme and/or triple helix methods. In another embodiment, an increase in CD36 gene expression may be desired to compensate for a mutant or impaired gene in an HSP-CD36 mediated pathway, and to ameliorate the symptoms of an HSP-CD36 related disorder.

Among the compounds that may exhibit the ability to modulate the activity, CD36 expression or synthesis of CD36 gene, including the ability to ameliorate the symptoms of an HSP-related disorder are antisense, ribozyme, and triple helix molecules. Such molecules may be designed to reduce or inhibit either unimpaired, or if appropriate, mutant target gene activity. Techniques for the production and use of such molecules are well known to those of skill in the art.

Antisense RNA and DNA molecules act to directly block the translation of mRNA by hybridizing to targeted mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required.

A sequence “complementary” to a portion of an RNA, as referred to herein, means a sequence having sufficient complementarity to be able to hybridize with the RNA, forming a table duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

In one embodiment, oligonucleotides complementary to non-coding regions of CD36 gene could be used in an antisense approach to inhibit translation of endogenous CD36 mRNA. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

In an embodiment of the present invention, oligonucleotides complementary to the nucleic acids encoding the HSP receptor ligand binding domain are used.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86, 6553-6556; Lemaitre et al., 1987, Proc. Natl. Acad. Sci. 84, 648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., 1988, BioTechniques 6, 958-976) or intercalating agents (see, e.g., Zon, 1988, Pharm. Res. 5, 539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate (S-ODNs), a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15, 6625-6641). The oligonucleotide is a 2′-0-methylribonucleotide (Inoue et al., 1987, Nucl. Acids Res. 15, 6131-6148), or a chimeric RNA-DNA analogue (Inoue et al., 1987, FEBS Lett. 215, 327-330).

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16, 3209), methylphosphonate oligonucleotides can be prepared by use of controlled pore glass polymer supports (Sarin et al., 1988, Proc. Natl. Acad. Sci. U.S.A. 85, 7448-7451), etc.

While antisense nucleotides complementary to the target gene coding region sequence could be used, those complementary to the transcribed, untranslated region are most preferred.

In one embodiment of the present invention, gene expression downregulation is achieved because specific target mRNAs are digested by RNAse H after they have hybridized with the antisense phosphorothioate oligonucleotides (S-ODNs). Since no rules exist to predict which antisense S-ODNs will be more successful, the best strategy is completely empirical and consists of trying several antisense S-ODNs. Antisense phosphorothioate oligonucleotides (S-ODNs) will be designed to target specific regions of mRNAs of interest. Control S-ODNs consisting of scrambled sequences of the antisense S-ODNs will also be designed to assure identical nucleotide content and minimize differences potentially attributable to nucleic acid content. All S-ODNs can be synthesized by Oligos Etc. (Wilsonville, Oreg.). In order to test the effectiveness of the antisense molecules when applied to cells in culture, such as assays for research purposes or ex vivo gene therapy protocols, cells will be grown to 60-80% confluence on 100 mm tissue culture plates, rinsed with PBS and overlaid with lipofection mix consisting of 8 ml Opti-MEM, 52.8 μl Lipofectin, and a final concentration of 200 nM S-ODNs. Lipofections will be carried out using Lipofectin Reagent and Opti-MEM (Gibco BRL). Cells will be incubated in the presence of the lipofection mix for 5 hours. Following incubation the medium will be replaced with complete DMEM. Cells will be harvested at different time points post-lipofection and protein levels will be analyzed by Western blot.

Antisense molecules should be targeted to cells that express the target gene, either directly to the subject in vivo or to cells in culture, such as in ex vivo gene therapy protocols. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced e.g., such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bemoist and Chambon, 1981, Nature 290, 304-310), the promoter contained in the 3′ long terminal repeat of Rous sarcoma virus (Yamamoto et al., 1980, Cell 22, 787-797), the herpes thymidine kinase promoter (Wagner et al., 1981, Proc. Natl. Acad. Sci. U.S.A. 78, 1441-1445), the regulatory sequences of the metallothionein gene (Brinster et al., 1982, Nature 296, 39-42), etc. Any type of plasmid, cosmid, YAC or viral vector can be used to prepare the recombinant DNA construct which can be introduced directly into the tissue site. Alternatively, viral vectors can be used that selectively infect the desired tissue, in which case administration may be accomplished by another route (e.g., systemically).

Ribozyme molecules designed to catalytically cleave target gene mRNA transcripts can also be used to prevent translation of target gene mRNA and, therefore, expression of target gene product. (See, e.g., PCT International Publication WO90/11364, published Oct. 4, 1990; Sarver et al., 1990, Science 247, 1222-1225). In an embodiment of the present invention, oligonucleotides which hybridize to the HSP receptor gene are designed to be complementary to the nucleic acids encoding the HSP receptor ligand binding domain.

Ribozymes are enzymatic RNA molecules capable of catalyzing the specific cleavage of RNA. (For a review, see Rossi, 1994, Current Biology 4, 469-471). The mechanism of ribozyme action involves sequence specific hybridization of the ribozyme molecule to complementary target RNA, followed by an endonucleolytic cleavage event. The composition of ribozyme molecules must include one or more sequences complementary to the target gene mRNA, and must include the well known catalytic sequence responsible for mRNA cleavage. For this sequence, see, e.g., U.S. Pat. No. 5,093,246, which is incorporated herein by reference in its entirety.

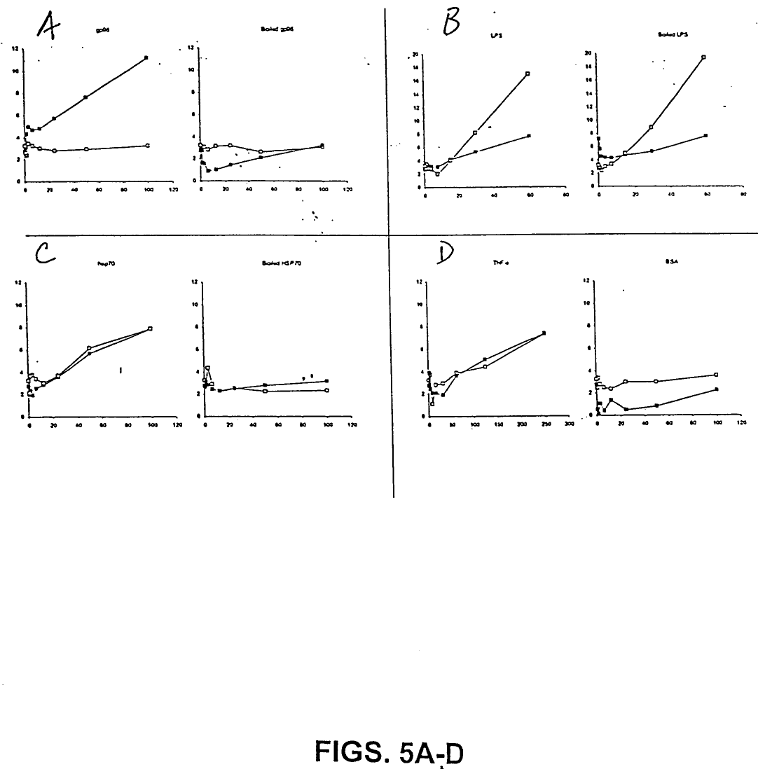

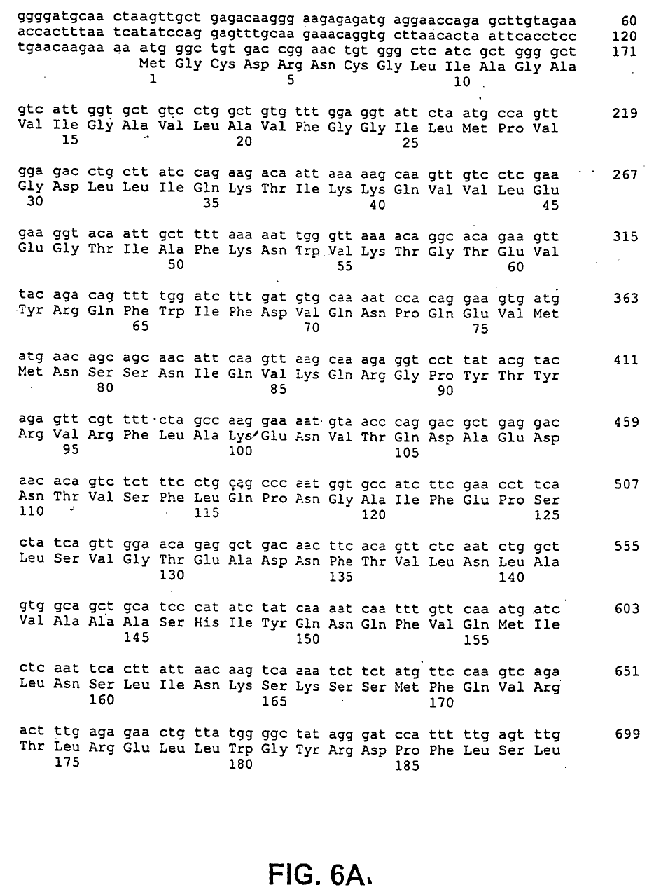

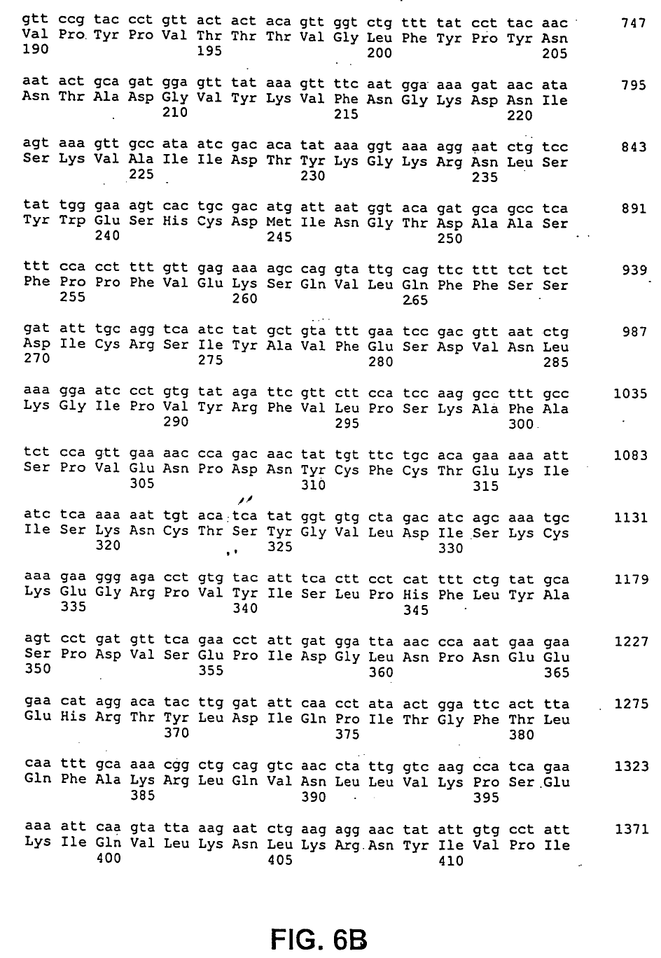

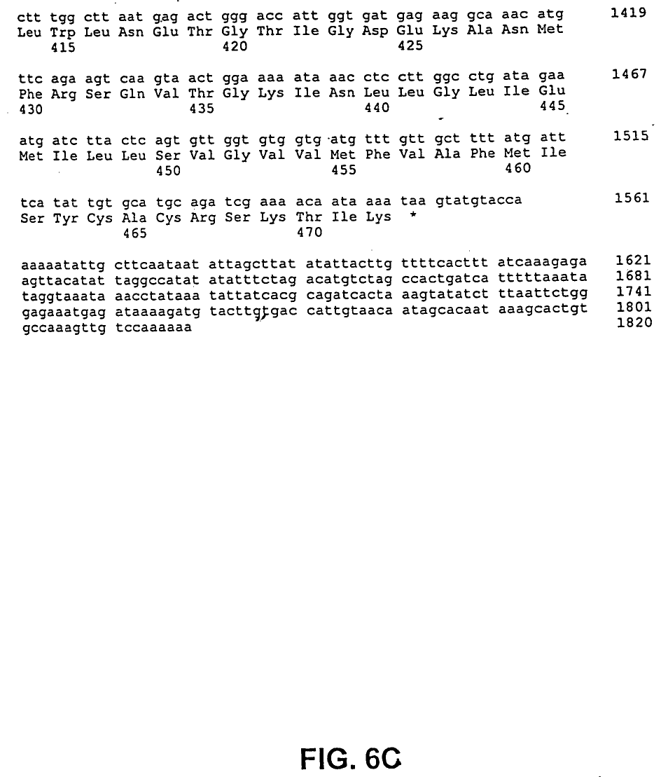

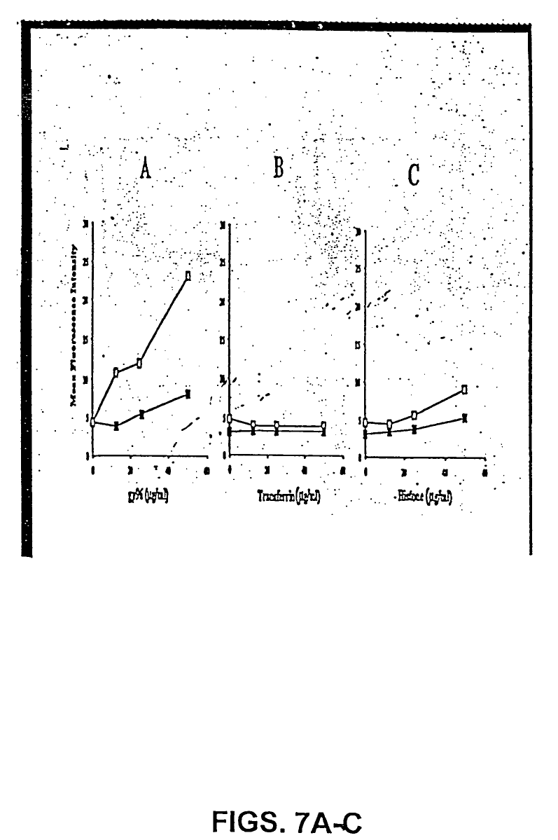

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Myers, 1995, Molecular Biology and Biotechnology: A Comprehensive Desk Reference, VCH Publishers, New York, (see especially FIG. 4, p. 833) and in Haseloff & Gerlach, 1988, Nature, 334, 585-591, which is incorporated herein by reference in its entirety.

Preferably the ribozyme is engineered so that the cleavage recognition site is located near the 5′ end of the target gene mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA) and that has been extensively described by Thomas Cech and collaborators (Zaug et al., 1984, Science, 224, 574-578; Zaug and Cech, 1986, Science, 231, 470-475; Zaug et al., 1986, Nature, 324, 429-433; published International patent application No. WO 88/04300 by University Patents Inc.; Been & Cech, 1986, Cell, 47, 207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in the target gene.

As in the antisense approach, the ribozymes can be composed of modified oligonucleotides (e.g., for improved stability, targeting, etc.) and should be delivered to cells that express the target gene in vivo. A preferred method of delivery involves using a DNA construct “encoding” the ribozyme under the control of a strong constitutive pol III or pol II promoter, so that transfected cells will produce sufficient quantities of the ribozyme to destroy endogenous target gene messages and inhibit translation. Because ribozymes, unlike antisense molecules, are catalytic, a lower intracellular concentration is required for efficiency.

Endogenous target gene expression can also be reduced by inactivating or “knocking out” the target gene or its promoter using targeted homologous recombination (e.g., see Smithies et al., 1985, Nature 317, 230-234; Thomas & Capecchi, 1987, Cell 51, 503-512; Thompson et al., 1989, Cell 5, 313-321; each of which is incorporated by reference herein in its entirety). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited modifications to ES (embryonic stem) cells can be used to generate animal offspring with an inactive target gene (e.g., see Thomas & Capecchi, 1987 and Thompson, 1989, supra). However this approach can be adapted for use in humans provided the recombinant DNA constructs are directly administered or targeted to the required site in vivo using appropriate viral vectors.

Alternatively, endogenous target gene expression can be reduced by targeting deoxyribonucleotide sequences complementary to the regulatory region of the target gene (i.e., the target gene promoter and/or enhancers) to form triple helical structures that prevent transcription of the target gene in target cells in the body. (See generally, Helene, 1991, Anticancer Drug Des., 6(6), 569-584; Helene et al., 1992, Ann. N.Y. Acad. Sci., 660, 27-36; and Maher, 1992, Bioassays 14(12), 807-815).

Nucleic acid molecules to be used in triple helix formation for the inhibition of transcription should be single stranded and composed of deoxyribonucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC⁺ triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

In instances wherein the antisense, ribozyme, and/or triple helix molecules described herein are utilized to inhibit mutant gene expression, it is possible that the technique may so efficiently reduce or inhibit the transcription (triple helix) and/or translation (antisense, ribozyme) of mRNA produced by normal target gene alleles that the possibility may arise wherein the concentration of normal target gene product present may be lower than is necessary for a normal phenotype. In such cases, to ensure that substantially normal levels of target gene activity are maintained, therefore, nucleic acid molecules that encode and express target gene polypeptides exhibiting normal target gene activity may, be introduced into cells via gene therapy methods such as those described, below, in Section 5.6.3 that do not contain sequences susceptible to whatever antisense, ribozyme, or triple helix treatments are being utilized. Alternatively, in instances whereby the target gene encodes an extracellular protein, it may be preferable to co-administer normal target gene protein in order to maintain the requisite level of target gene activity.

Anti-sense RNA and DNA, ribozyme, and triple helix molecules of the invention may be prepared by any method known in the art for the synthesis of DNA and RNA molecules, as discussed above. These include techniques for chemically synthesizing oligodeoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule.

Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

5.6.3 Gene Replacement Therapy

With respect to an increase in the level of normal CD36 gene expression and/or CD36 gene product activity, CD36 gene nucleic acid sequences can, for example, be utilized for the treatment of immune disorders resulting in proliferative disorders such as cancer. Such treatment can be administered, for example, in the form of gene replacement therapy. Specifically, one or more copies of a normal CD36 gene or a portion of CD36 gene that directs the production of a CD36 gene product exhibiting normal CD36 gene function, may be inserted into the appropriate cells within a patient, using vectors that include, but are not limited to adenovirus, adeno-associated virus, and retrovirus vectors, in addition to other particles that introduce DNA into cells, such as liposomes.

Gene replacement therapy techniques should be capable of delivering CD36 gene sequences to cell types that express the HSP receptor within patients. Thus, in one embodiment, techniques that are well known to those of skill in the art (see, e.g., PCT Publication No. WO89/10134, published Apr. 25, 1988) can be used to enable CD36 gene sequences to be delivered to developing cells of the myeloid lineage, for example, to the bone marrow. In another specific embodiment, gene replacement can be accomplished using macrophages in vitro, and delivered to a patient using the techniques of adoptive immunotherapy.

In another embodiment, techniques for delivery involve direct administration of such gene sequences to the site of the cells in which CD36 gene sequences are to be expressed, e.g., directly at the site of the tumor.

Additional methods that may be utilized to increase the overall level of CD36 gene expression and/or CD36 gene product activity include the introduction of appropriate CD36-expressing cells, preferably autologous cells, into a patient at positions and in numbers that are sufficient to ameliorate the symptoms of a CD36 disorder. Such cells may be either recombinant or non-recombinant.

Among the cells that can be administered to increase the overall level of CD36 gene expression in a patient are cells that normally express CD36 gene.

Alternatively, cells, preferably autologous cells, can be engineered to express CD36 gene sequences, and may then be introduced into a patient in positions appropriate for the amelioration of the symptoms of a CD36 disorder or a proliferative or viral disease, e.g., cancer and tumorigenesis. Alternately, cells that express an unimpaired CD36 gene and that are from a MHC matched individual can be utilized, and may include, for example, brain cells. The expression of CD36 gene sequences is controlled by the appropriate gene regulatory sequences to allow such expression in the necessary cell types. Such gene regulatory sequences are well known to the skilled artisan. Such cell-based gene therapy techniques are well known to those skilled in the art, see, e.g., Anderson, U.S. Pat. No. 5,399,349.

When the cells to be administered are non-autologous cells, they can be administered using well known techniques that prevent a host immune response against the introduced cells from developing. For example, the cells may be introduced in an encapsulated form which, while allowing for an exchange of components with the immediate extracellular environment, does not allow the introduced cells to be recognized by the host immune system.

5.6.4 Delivery of Soluble Polypeptides

Genetically engineered cells that express soluble ECDs or fusion proteins, e.g., fusion Ig molecules can be administered in vivo where they may function as “bioreactors” that deliver a supply of the soluble molecules. Such soluble CD36 polypeptides and fusion proteins, when expressed at appropriate concentrations, should neutralize or “mop up” HSPs or other native ligand for CD36, and thus act as inhibitors of activity and may therefore be used to treat HSP-CD36 related disorders and diseases, such as autoimmune disorders, proliferative disorders, and infectious diseases.

5.6.5 Delivery of Dominant Negative Mutants

In another embodiment of the invention, dominant negative mutants (“dominant negatives”) may be used therapeutically to block the immune response to an HSP-antigen complex, e.g., to treat an auto-immune disorder. In general, such dominant-negatives are mutants which, when expressed, interact with ligand (i.e., HSP-antigenic molecule complex), but lack one or more functions, i.e. growth factor production and/or signaling functions, of normal CD36. Such mutants interfere with the function of normal CD36 in the same cell or in a different cell, e.g. by titration of HSP-peptide complexes from the wild type receptor. Such a mutation, for example, can be one or more point mutation(s), a deletion, insertion, or other mutation in either the extracellular, the transmembrane or intracellular domains.

Expression of a such a dominant negative mutation in a cell can block uptake of ligand by normal functional receptors in the same or neighboring cells by titrating out the amount of available ligand. Thus, a recombinant antigen presenting cell expressing such a dominant negative can be used to titrate out HSP-antigenic molecule complexes when administered to a patient in need of treatment for an autoimmune disorder.

5.7 Target Autoimmune Diseases

Autoimmune diseases that can be treated by the methods of the present invention include, but are not limited to, insulin dependent diabetes mellitus (i.e., IDDM, or autoimmune diabetes), multiple sclerosis, systemic lupus erythematosus, Sjogren's syndrome, scleroderma, polymyositis, chronic active hepatitis, mixed connective tissue disease, primary biliary cirrhosis, pernicious anemia, autoimmune thyroiditis, idiopathic Addison's disease, vitiligo, gluten-sensitive enteropathy, Graves' disease, myasthenia gravis, autoimmune neutropenia, idiopathic thrombocytopenia purpura, rheumatoid arthritis, cirrhosis, pemphigus vulgaris, autoimmune infertility, Goodpasture's disease, bullous pemphigoid, discoid lupus, ulcerative colitis, and dense deposit disease. The diseases set forth above, as referred to herein, include those exhibited by animal models for such diseases, such as, for example non-obese diabetic (NOD) mice for IDDM and experimental autoimmune encephalomyelitis (EAE) mice for multiple sclerosis.

The methods of the present invention can be used to treat such autoimmune diseases by reducing or eliminating the immune response to the patient's own (self) tissue, or, alternatively, by reducing or eliminating a pre-existing autoimmune response directed at tissues or organs transplanted to replace self tissues or organs damaged by the autoimmune response.

5.8 Target Infectious Diseases

The infectious diseases that can be treated or prevented using the methods and compositions of the present invention include those caused by intracellular pathogens such as viruses, bacteria, protozoans, and intracellular parasites. Viruses include, but are not limited to viral diseases such as those caused by hepatitis type B virus, parvoviruses, such as adeno-associated virus and cytomegalovirus, papovaviruses such as papilloma virus, polyoma viruses, and SV40, adenoviruses, herpes viruses such as herpes simplex type I (HSV-I), herpes simplex type II (HSV-II), and Epstein-Barr virus, poxviruses, such as variola (smallpox) and vaccinia virus, RNA viruses, including but not limited to human immunodeficiency virus type I (HIV-I), human immunodeficiency virus type II (HIV-II), human T-cell lymphotropic virus type I (HTLV-I), and human T-cell lymphotropic virus type II (HTLV-II); influenza virus, measles virus, rabies virus, Sendai virus, picornaviruses such as poliomyelitis virus, coxsackieviruses, rhinoviruses, reoviruses, togaviruses such as rubella virus (German measles) and Semliki forest virus, arboviruses, and hepatitis type A virus.

In another embodiment, bacterial infections can be treated or prevented such as, but not limited to disorders caused by pathogenic bacteria including, but not limited to, Streptococcus pyogenes, Streptococcus pneumoniae, Neisseria gonorrhoea, Neisseria meningitidis, Corynebacterium diphtheriae, Clostridium botulinum, Clostridium perfringens, Clostridium tetani, Haemophilus influenzae, Klebsiella pneumoniae, Klebsiella ozaenae, Klebsiella rhinoscleromotis, Staphylococcus aureus, Vibrio cholerae, Escherichia coli, Pseudomonas aeruginosa, Campylobacter (Vibrio) fetus, Campylobacter jejuni, Aeromonas hydrophila, Bacillus cereus, Edwardsiella tarda, Yersinia enterocolitica, Yersinia pestis, Yersinia pseudotuberculosis, Shigella dysenteriae, Shigella flexneri, Shigella sonnei, Salmonella typhiimurium, Salmonella typhii, Treponema pallidum, Treponema pertenue, Treponema carateneum, Borrelia vincentii, Borrelia burgdorferi, Leptospira icterohemorrhagiae, Mycobacterium tuberculosis, Toxoplasma gondii, Pneumocystis carinii, Francisella tularensis, Brucella abortus, Brucella suis, Brucella melitensis, Mycoplasma spp., Rickettsia prowazeki, Rickettsia tsutsugumushi, Chlamydia spp., and Helicobacter pylori.

In another preferred embodiment, the methods can be used to treat or prevent infections caused by pathogenic protozoans such as, but not limited to, Entomoeba histolytica, Trichomonas tenas, Trichomonas hominis, Trichomonas vaginalis, Trypanosoma gambiense, Trypanosoma rhodesiense, Trypanosoma cruzi, Leishmania donovani, Leishmania tropica, Leishmania braziliensis, Pneumocystis pneumonia, Plasmodium vivax, Plasmodium falciparum, and Plasmodium malaria.

5.9 Target Proliferative Cell Disorders

With respect to specific proliferative and oncogenic disease associated with HSP-CD36 activity, the diseases that can be treated or prevented by the methods of the present invention include, but are not limited to: human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenström's macroglobulinemia, and heavy chain disease.

Diseases and disorders involving a deficiency in cell proliferation or in which cell proliferation is desired for treatment or prevention, and that can be treated or prevented by inhibiting CD36 function, include but are not limited to degenerative disorders, growth deficiencies, hypoproliferative disorders, physical trauma, lesions, and wounds; for example, to promote wound healing, or to promote regeneration in degenerated, lesioned or injured tissues, etc.

5.10 Pharmaceutical Preparations and Methods of Administration

The compounds that are determined to affect CD36 gene expression or gene product activity can be administered to a patient at therapeutically effective doses to treat or ameliorate a cell proliferative disorder. A therapeutically effective dose refers to that amount of the compound sufficient to result in amelioration of symptoms of such a disorder.

5.10.1 Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

5.10.2 Formulations and Use

Pharmaceutical compositions for use in accordance with the present invention may be formulated in conventional manner using one or more physiologically acceptable carriers or excipients.

Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration.

### 6. EXAMPLE

### Identification of CD36 as an HSP Receptor

6.1 Introduction

The Example presented herein describes the successful identification of an interaction between gp96 and the CD36 receptor present in macrophages and dendritic cells. The experiments presented herein form the basis for isolating CD36 receptor polypeptides and for the screening, diagnostic, and therapeutic methods of the present invention.

The Applicants of the present invention noted that certain observations were inconsistent with a “direct transfer” model of HSP-chaperoned peptide antigen presentation. First, the immunogenicity of HSP preparations is dependent on the presence of functional phagocytic cells but not B cells or other nonprofessional antigen-presenting cells, (Udono and Srivastava, 1993, supra; Suto and Srivastava, 1995, supra), whereas free peptides can sensitize all cell types. Second, extremely small quantities of HSP-peptide complexes were effective in eliciting specific immunity, i.e., gp96-chaperoned peptides are several hundred times as effective as free peptides in sensitizing macrophages for CTL recognition, suggesting the possibility of a specific uptake mechanism. Third, gp96-chaperoned peptides elicited an MHC I response that was not limited by the size of peptide. Finally, the processing of gp96-peptide complexes in macrophage was found to be sensitive to Brefeldin A (BFA), which blocks transport through the Golgi apparatus, suggesting that processing occurred through an intercellular mechanism. These observations led to the hypothesis that HSP-chaperoned peptides may be processed internally and re-presented by MHC class I molecules on the cell surfaces of macrophages (Suto and Srivastava, 1995, supra). There is also the hypothesis that the mannose receptor is used in the uptake of gp96 but no mechanism has been proposed for the non-glycosylated HSPs, such as HSP70 (Ciupitu et al., 1998, J. Exp. Med., 187: 685-691). Others suggested that a novel intracellular trafficking pathway may be involved for the transport of peptides from the extracellular medium into the lumen of ER) Day et al., 1997, Proc. Natl. Acad. Sci. 94:8065-8069; Nicchitta, 1998, Curr. Opin. in Immunol. 10:103-109). Further suggestions include the involvement of phagocytes which (a) possess an ill-defined pathway to shunt protein from the phagosome into the cytosol where it would enter the normal class I pathway; (b) digest ingested material in lysosomes and regurgitate peptides for loading on the surface to class I molecules (Bevan, 1995, J. Exp. Med. 192:639-41). The discovery of a receptor for heat shock protein helps to resolve the paradox of how extracellular antigenic peptides complexed to HSPs can stimulate the immune response. In addition, cell surface receptors can have other functions, such as inducing a signal transduction pathway. CD36, as disclosed herein, has been identified as such a receptor.

6.2 Materials and Methods

Macrophage isolation. Wild type and CD36 null mice were inoculated with 0.2 ml of pristane. Five days later, macrophages were extracted from the peritoneal lavage and plated or 3-4 hours using standard cell culture techniques.

Dendritic cell isolation. Bone marrow cells were extracted from the femur of wild type and CD36 null mice. Cells were incubated in the presence of GM-CSF for 6 days. After 6 days, the cells were replated for 1 extra day to select for non-adherent dendritic cells.

MCP-1 chemokine assay. 1×10⁵ of wild type and CD36 null dendritic cells or macrophages were prepared per well in 96 well plates and incubated for 20 hours with increasing concentrations of gp96, LPS, HSP70, TNF-α, and BSA. Supernatants were harvested and analyzed by MCP-1 sandwich ELISA (R&D Systems) as per manufacturer's instructions and the optical density was determined at 450 nm.

Nitric oxide assay. Wild type and CD36 null macrophage and dendritic cells were prepared as above and incubated at 1×10⁵ cells in 96 well plates with increasing concentrations of gp96, LPS, HSP70, TNF-α, and BSA. The supernatants were harvested after 20 hours of incubation and analyzed by enzymatic Greiss assay (Calbiochem) as per manufacturer's instructions. The optical density was determined at 540 nm.

Binding assay. HEK293 cells were transfected using FuGENE 6 (Roche Diagnostics) and a plasmid construct containing the CD36 cDNA (CD36 cDNA cloned into pCDNA 3.1 expression vector). After a two day period to allow expression of the transgene, cells were harvested and incubated with varying concentrations of fluorescent protein probes (GP96-FITC, Histone FITC, AcLDL-DiI, Ova-FITC, and BSA (fatty-acid free)-FITC). Some experiments were performed with the addition of only an anti-CD36 monoclonal antibody and then a second fluorescently labelled anti-mouse antibody was added to detect CD36 expression. Cells were analyzed by flow cytometry. Low and high CD36 expressing populations were gated and analyzed for binding of proteins indicated. Mean fluorescence intensity of each population was plotted. Competitive inhibition experiments were performed by preincubating the cells at 4° C. with 50 ug/ml of normal/modified LDL or with 25 ug/ml of anti-CD36 antibody.

6.3 Results

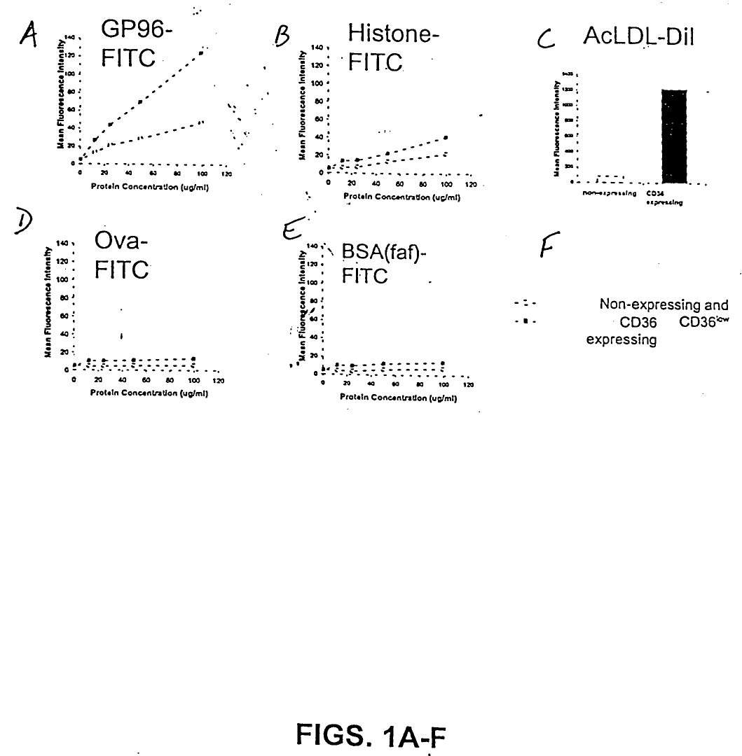

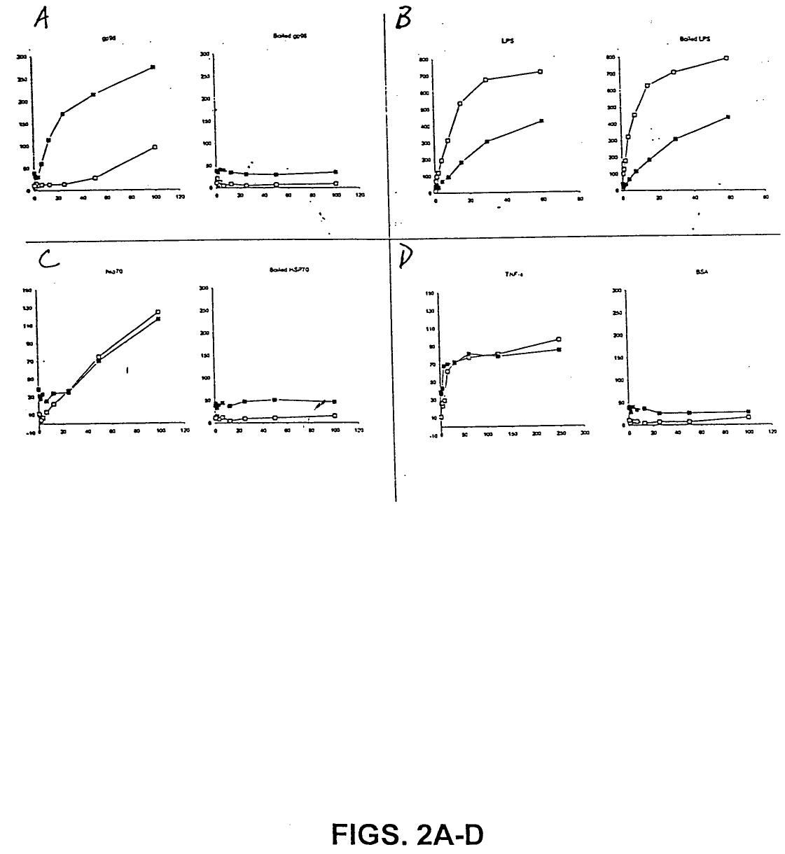

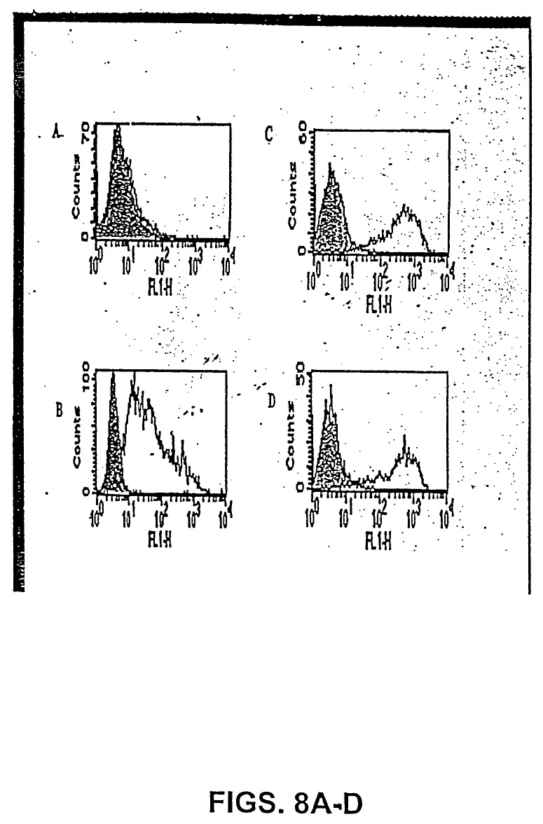

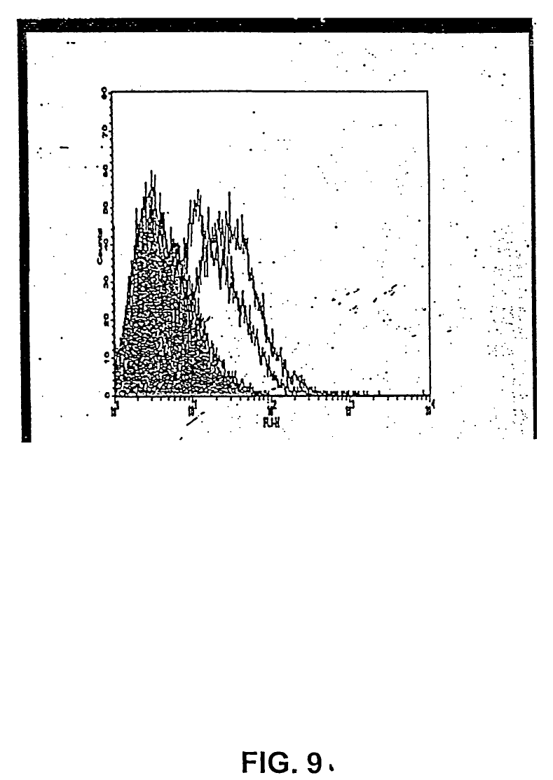

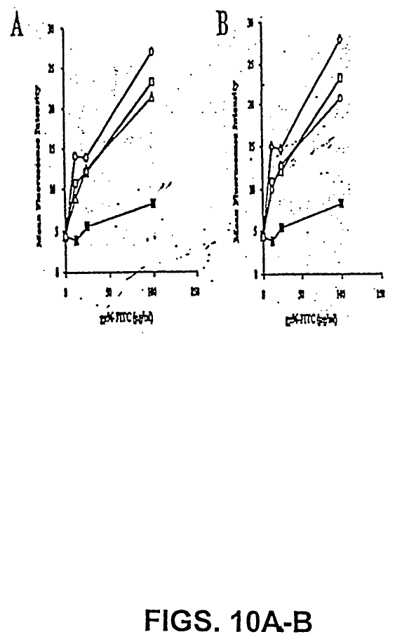

Determination that gp96 binds CD36. Homogenous preparations of gp96 were coupled to FITC and the gp96-FITC was used to stain HEK293 cells and HEK293 CD36 transfectants. The CD36 expressing cells bound to Gp96-FITC whereas the non-expressing and CD36^{low} did not bind (FIG. 1A). This binding was specific for GP96 as shown by the lack of any staining of the HEK293 cells to Histone-FITC, Ova-FITC, BSA(faf)-FITC (FIGS. 1B-D). The positive control, acetylated low density lipoprotein, stained the CD36 expressing cells and did not stain the non-expressing CD36^{low} cells (FIG. 1E) demonstrating that the transfected HEK293 cells express a functional CD36 protein and gp96 binding is specific to CD36. To confirm that the transfected HEK293 cells were properly expressing CD36, HEK293 transfected cells and mock transfected cells were incubated with a mouse anti-CD36 antibody and then stained with an anti-mouse-FITC antibody. The mock transfected cells did not stain (FIG. 8A) whereas the transfected cells were stained (FIGS. 8C and 8D). When gp96-FITC was incubated with wild type and CD36 null macrophages, a 52% loss of binding to gp96 is exhibited by the CD36 null cells as compared to wild type cells (FIG. 9). When LDL or anti-CD36 is used as a competitive inhibitor for gp96 binding, no inhibition occurs (FIGS. 10A and 10B). This indicates that gp96 does not bind to CD36 at a.a. 155-183 or a.a. 28-93, the respective binding regions of the anti-CD36 antibody and LDL, under the conditions tested.

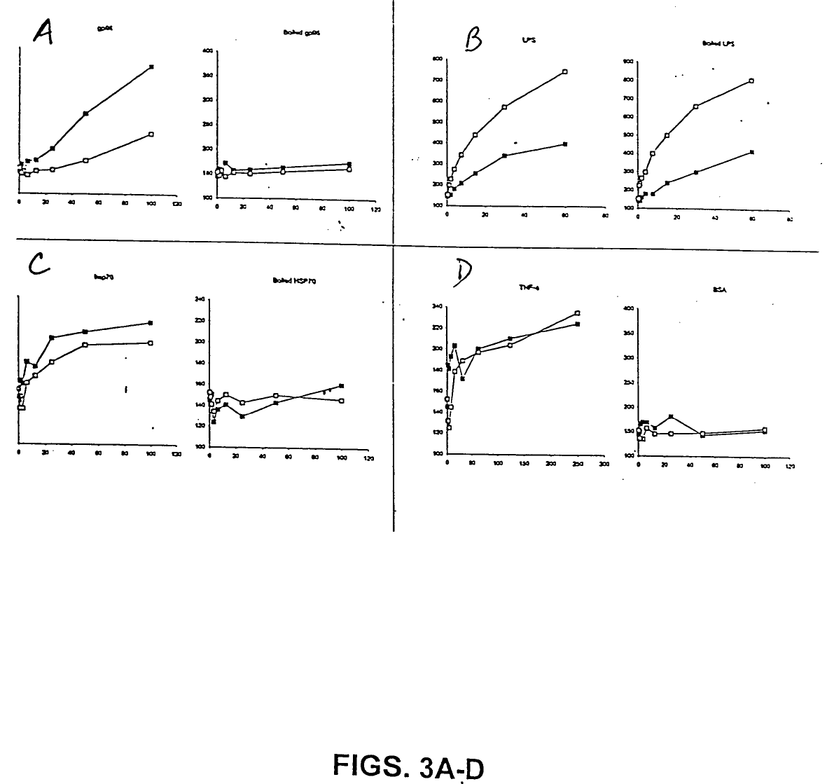

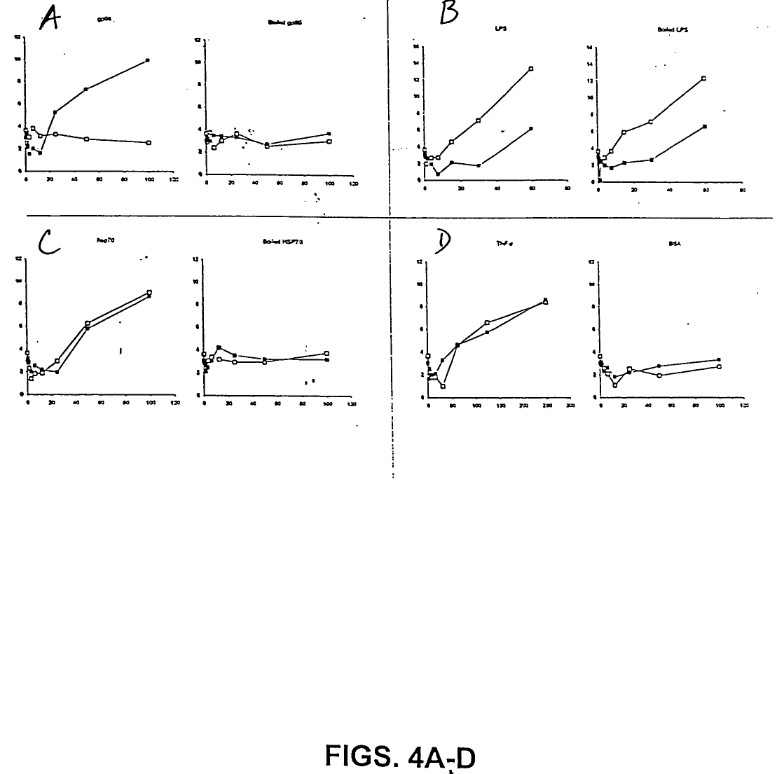

Gp96 binding to CD36 induces chemokine production in macrophages and dendritic cells. Both wild type and CD36 null mice were injected with 0.2 ml pristane. Macrophages were extracted from such mice. Dendritic cells were extracted and cultured from bone marrow of non-pristane injected mice. Macrophages and dendritic cells were incubated for 20 hours in the presence of increasing concentrations of various native and denatured proteins (boiled for 30 minutes). The level of chemokine production from the mice was measured using standard ELISA sandwich assay. Native gp96 induced a strong production of chemokine in both wild type macrophages and dendritic cells as compared to the CD36 null population (FIGS. 2A and 3A). Boiled gp96 did not induce any chemokine production in either the wild type macrophages and dendritic cells or the CD36 null macrophages and dendritic cells (FIGS. 2A and 3A). LPS induced chemokine production but this was not due to CD36 because the level of chemokine production is greater in the CD36 null cells than in the wild type cells (FIGS. 2B and 3B). In addition, the level of chemokine is the same in both the native and boiled samples of LPS (FIGS. 2B and 3B). Both the native and boiled HSP70 induced the same amount of chemokine in macrophages and dendritic cells in both the wild type and CD36 null cells demonstrating that this induction is not related to CD36 (FIGS. 2C and 3C). TNF-α and BSA induced an equal amount of chemokine production in both the wild type and CD36 null cells also demonstrating that they do not act in chemokine production through CD36 (FIGS. 2D and 3D).

Gp96 binding to CD36 induces nitric oxide production in macrophages and dendritic cells. Identical experiments were performed to determine the level of nitric oxide produced by wild type and CD36 null cells. Macrophages and dendritic cells were incubated for 20 hours in the presence of increasing concentrations of various native and denatured proteins (boiled for 30 minutes) and the level of nitric oxide production was determined by the enzymatic Greiss assay. Native gp96 induced a strong nitric oxide production in both macrophage and dendritic wild type cells compared to the CD36 null cells (FIGS. 4A and 5A). Boiled gp96 did not induce any nitric oxide production in either the wild type macrophages and dendritic cells or the CD36 null macrophages and dendritic cells (FIGS. 4A and 5A). LPS induced nitric oxide production but once again this was not due to CD36 because the level of nitric oxide production is greater in the CD36 null cells than in the wild type cells (FIGS. 4B and 5B). Both the native and boiled HSP70 induced the same amount of nitric oxide in macrophages and dendritic cells in both the wild type and CD36 null cells demonstrating that this induction is not related to CD36 (FIGS. 4C and 5C). TNF-α and BSA induced an equal amount of nitric oxide production in both the wild type and CD36 null cells also demonstrating that the induction of nitric oxide in CD36 expressing cells is due to the gp96-CD36 interaction (FIGS. 2D and 3D).

6.4 Discussion

The studies reported here show that the heat shock protein gp96 is an additional ligand for the CD36 receptor. The human gp96-coding gene has been mapped previously by us at chromosome 12 (q24.2→q24.3) (Maki et al., 1993, Somatic Cell Mol. Gen. 19:73-81).

As shown here, the gp96-CD36 receptor interaction provides a new type of function for CD36 receptor, a function of a sensor, not only of the extracellular environment with its previously known plasma-based. ligands, but also a sensor of the intracellular milieu as well. HSPs such as gp96 are obligate intracellular molecules and are released into the extracellular milieu only under conditions of necrotic (but not apoptotic) cell death. Thus, the CD36 receptor may act as a sensor for necrotic cell death, just as the recently identified phosphatidyl serine-binding protein act as sensors of apoptotic cell death and receptors for apoptotic cells (Savill et al., 1992, J. Clin. Invest.90:1513-1522; Fadok et al., 2000, Nature 405:85-90). Interaction of the macrophages with the apoptotic cells leads to a down-regulation of the inflammatory cytokines such as TNF (Fadok et al., 2000, supra), while gp96-APC interaction induces a signal transduction pathway resulting in stimulation of antigen-specific T cells (Suto and Srivastava, 1995, supra) and, in addition, secretion of pro-inflammatory cytokines such as TNF, GM-CSF and IL-12.

It is possible, therefore, that the CD36 receptor renders it possible for the APCs to sample (i) the extracellular milieu of the blood through CD36 and other plasma ligands and (ii) the intracellular milieu of the tissues through HSPs, particularly of the gp96 family. The former permits APCs to implement their primordial phagocytic function, while the latter allows them to execute its innate and adaptive immunological functions. Viewed in another perspective, recognition of apoptotic cells by APCs through phophatidyl serine, leads to anti-inflammatory signals, while interaction of the APC with necrotic cells through CD36 receptor leads to pro-inflammatory immune responses (see Srivastava et al., 1998, Immunity 8: 657-665).

The invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

All references cited herein, including patent applications, patents, and other publications, are incorporated by reference herein in their entireties for all purposes The invention is not to be limited in scope by the specific embodiments described which are intended as single illustrations of individual aspects of the invention, and functionally equivalent methods and components are within the scope of the invention. Indeed various modifications of the invention, in addition to those shown and described herein will become apparent to those skilled in the art from the foregoing description and accompanying drawings. Such modifications are intended to fall within the scope of the appended claims.

All references cited herein, including patent applications, patents, and other publications, are incorporated by reference herein in their entireties for all purposes.

What is claimed is:

1. A method for identifying a compound that modulates an HSP-CD36 mediated process, comprising:
(a) contacting a test compound with a heat shock protein and CD36; and
(b) measuring the level of CD36 activity or expression,
such that if the level of activity or expression measured in (b) differs from the level of CD36 activity in the absence of the test compound, then a compound that modulates an HSP-CD36-mediated process is identified.

2. The method of claim 1, in which the compound identified is an antagonist which interferes with the interaction of the heat shock protein with CD36, further comprising the step of:
(c) determining whether the level interferes with the interaction of the heat shock protein and CD36.

3. The method of claim 1, in which the test compound is an antibody specific for CD36.

4. The method of claim 1, in which the test compound is an antibody specific for a heat shock protein.

5. The method of claim 1, in which the test compound is a small molecule.

6. The method of claim 1, in which the test compound is a peptide.

7. The method of claim 6, in which the peptide comprises at least 5 consecutive amino acids of CD36.

8. The method of claim 6, in which the peptide comprises at least 10 consecutive amino acids of CD36.

9. The method of claim 6, in which the peptide comprises at least 5 consecutive amino acids of a heat shock protein sequence.

10. The method of claim 1, in which the compound is an agonist which enhances the interaction of the heat shock protein with CD36.

11. The method of claim 1 in which the HSP-CD36 mediated process affects an autoimmune disorder, a disease or disorder involving disruption of signal transducer activity, a disease or disorder involving cytokine clearance or inflammation, a proliferative disorder, a viral disorder or other infectious disease, hypercholesterolemia, Alzheimer's disease, diabetes, or osteoporosis.

12. A method for identifying a compound that modulates an HSP-CD36 mediated process, comprising:
(a) contacting a test compound with a heat shock protein and a CD36-expressing cell; and
(b) measuring the level of CD36 activity or expression in the cell,
such that if the level of activity or expression measured in (b) differs from the level of CD36 activity in the absence of the test compound, then a compound that modulates an HSP-CD36-mediated process is identified.

13. The method of claim 1 or 12 wherein CD36 activity measured is the ability to interact with a heat shock protein.

14. The method of claim 12 wherein the heat shock protein is non-covalently associated with an antigenic peptide and CD36 activity measured is the ability to induce signal transduction activity.

15. The method of claim 12 wherein the heat shock protein is non-covalently associated with an antigenic peptide and CD36 activity measured is the ability to stimulate a cytotoxic T cell response against the antigenic peptide.

16. A method for identifying a compound that modulates the binding of a heat shock protein to CD36, comprising:
(a) contacting a heat shock protein with CD36, or fragment, or analog, derivative or mimetic thereof, in the presence of a test compound; and
(b) measuring the amount of heat shock protein bound to CD36, or fragment, analog, derivative or mimetic thereof,
such that if the amount of bound heat shock protein measured in (b) differs from the amount of bound heat shock protein measured in the absence of the test compound, then a compound that modulates the binding of an HSP to CD36 is identified.

17. The method of claim 16 wherein CD36 contacted in step (a) is on a cell surface.

18. The method of claim 16 wherein CD36 is immobilized to a solid surface.

19. The method of claim 18, 72 wherein the solid surface is a microtiter dish.

20. The method of claim 16 wherein the amount of bound heat shock protein is measured by contacting the cell with a heat shock protein-specific antibody.

21. The method of claim 16 wherein the heat shock protein is labeled and the amount of bound heat shock protein is measured by detecting the label.

22. The method of claim 21 wherein the heat shock protein is labeled with a fluorescent label.

23. A method for identifying a compound that modulates heat shock protein-mediated signal transduction by CD36-expressing cells comprising:
(a) adding a test compound to a mixture of CD36-expressing cells and a complex consisting essentially of a heat shock protein noncovalently associated with an antigenic molecule, under conditions conducive to CD36-mediated signal transduction;
(b) measuring the level of stimulation by CD36-expressing cells,
such that if the level measured in (b) differs from the level of said stimulation in the absence of the test compound, then a compound that modulates heat shock protein-mediated signal transduction by CD36-expressing cells is identified.

24. The method of claim 23 in which the measuring stimulation of antigen-specific cytotoxic T cells by CD36-expressing cells of step (b) comprises:
(i) adding CD36-expressing cells formed in step (a) to T cells under conditions conducive to the activation of the T cells; and
(ii) comparing the level of activation of said cytotoxic T cells with the level of activation of T cells by a CD36-expressing cell formed in the absence of the test compound,
wherein an increase of decrease in level of T cell activation indicates that a compound that modulates heat shock protein-mediated signal transduction by CD36-expressing cells is identified.

25. The method of claim 23 wherein the signal transduction activity is the production nitric oxide.

26. The method of claim 23 wherein the signal transduction activity is the production of MCP-1 chemokine

27. The method of claim 1, 17, 71, or 23 in which the heat shock protein is gp96.

28. A method for detecting a heat shock protein-CD36 related disorder in a mammal comprising measuring the level of activity from an HSP-CD36 mediated process in a patient sample, such that if the measured level differs from the level found in clinically normal individuals, then a heat shock protein-CD36 related disorder is detected.

29. The method of claim 28 comprising contacting a sample derived from a patient with an antibody specific for CD36 under conditions such that immunospecific binding by the antibody.

30. The method of claim 28 comprising contacting a sample derived from a patient with an antibody specific for a heat shock protein under conditions conducive for immunospecific binding by the antibody.

31. The method of claim 28 comprising contacting a sample derived from a patient with an antibody specific for an HSP-CD36 complex under conditions conducive for immunospecific binding by the antibody.

32. A method for modulating an immune response comprising administering to a mammal a purified compound that modulates the interaction of a heat shock protein with CD36.

33. The method of claim 32, in which the compound is an agonist which enhances the interaction of the heat shock protein and CD36.

34. A method for treating an autoimmune disorder comprising administering to a mammal in need of such treatment a purified compound that interferes with the interaction of a heat shock protein with CD36.

35. The method of claim 34 in which the compound is an antagonist that interferes with the interaction between the heat shock protein and CD36.

36. The method of claim 35, in which the antagonist is an antibody specific for CD36.

37. The method of claim 35, in which the antagonist is an antibody specific for a heat shock protein.

38. The method of claim 35, in which the antagonist is a small molecule.

39. The method of claim 35, in which the antagonist is a peptide.

40. The method of claim 39, in which the peptide comprises at least 5 consecutive amino acids of CD36.

41. The method of claim 39, in which the peptide comprises at least 5 consecutive amino acids of a heat shock protein sequence.

42. The method of claim 39, in which the peptide comprises at least 10 consecutive amino acids of a heat shock protein sequence.

43. A method for treating an autoimmune disorder comprising administering to a mammal in need of such treatment a recombinant cell that expresses CD36 which decreases signal transducing activity by a functional CD36.

44. A method for increasing the immunopotency of a cancer cell or an infected cell comprising transforming said cell with a nucleic acid comprising a nucleotide sequence that (i) is operably linked to a promoter, and (ii) encodes a CD36 polypeptide.

45. A method for increasing the immunopotency of a cancer cell or an infected cell comprising:
(a) transforming said cell with a nucleic acid comprising a nucleotide sequence that (i) is operably linked to a promoter, and (ii) encodes a CD36 polypeptide, and
(b) administering said cell to an individual in need of treatment, so as to obtain an elevated immune response.

46. A recombinant cancer cell transformed with a nucleic acid comprising a nucleotide sequence that (i) is operably linked to a promoter, and (ii) encodes a CD36 polypeptide.

47. A recombinant infected cell transformed with a nucleic acid comprising a nucleotide sequence that (i) is operably linked to a promoter, and (ii) encodes a CD36 polypeptide.

48. The recombinant cell of claim 46 or 47 which is a human cell.

49. A kit, comprising in one or more containers: (a) an anti-CD36 antibody or a nucleic acid probe capable of hybridizing to a CD36 nucleic acid, (b) a purified heat shock protein, nucleic acid encoding a heat shock protein, or cell expressing a heat shock protein; and (c) instructions for use in detecting a heat shock protein-CD36 related disorder.

50. The kit of claim 49 wherein the antibody or nucleic acid probe is labeled with a detectable marker.

51. The kit of claim 49 further comprising a labeled CD36 polypeptide.

52. A kit, in one or more containers, comprising: (a) a purified heat shock protein, nucleic acid encoding a heat shock protein, or cell expressing a heat shock protein; and (b) a CD36 polypeptide, nucleic acid encoding a CD36 polypeptide, or cell expressing a CD36 polypeptide.

53. The kit of claim 52 in which CD36 polypeptide, nucleic acid encoding a CD36 polypeptide, or cell expressing a CD36 polypeptide is purified.

54. The kit of claim 52 further comprising instructions for use in treating an autoimmune disorder, an infectious disease, or a proliferative disorder.

55. A method for identifying a CD36 fragment capable of binding a heat shock protein, said method comprising:
(a) contacting a heat shock protein, or peptide-binding fragment thereof, with one or more CD36 fragments; and
(b) identifying a CD36 fragment which specifically binds to the heat shock protein, or peptide-binding fragment thereof.

56. A method for identifying a CD36 fragment capable of inducing an HSP-CD36-mediated process, said method comprising:
(a) contacting a heat shock protein with a cell expressing a CD36 fragment; and
(b) measuring the level of CD36 activity in the cell,
such that if the level of the HSP-CD36 mediated process or activity measured in (b) is greater than the level of CD36 activity in the absence of the CD36 fragment, then a CD36 fragment capable of inducing an HSP-CD36 mediated process is identified.

57. The method of claim 56 wherein CD36 activity measured is the ability to interact with the heat shock protein.

58. The method of claim 56 wherein the heat shock protein is non-covalently associated with an antigenic peptide and CD36 activity measured is the ability to stimulate signal transduction activity.

59. The method of claim 58 wherein the signal transduction activity is the production of nitric oxide.

60. The method of claim 58 wherein the signal transduction activity is the production of MCP-1 chemokine.

61. The method of claim 56 wherein the heat shock protein is non-covalently associated with an antigenic peptide and CD36 activity measured is the ability to stimulate a cytotoxic T cell response against the antigenic peptide.

62. A method for identifying a heat shock protein fragment capable of binding a CD36, said method comprising:
(a) contacting CD36 with one or more heat shock protein fragments; and
(b) identifying a heat shock protein fragment which specifically binds to CD36.

63. A method for identifying a heat shock protein fragment capable of inducing an HSP-CD mediated process, said method comprising:
(a) contacting a CD36 fragment with a cell expressing a heat shock protein; and
(b) measuring the level of CD36 activity in the cell,
such that if the level of the HSP-CD36 mediated process or activity measured in (b) is greater than the level of CD36 activity in the absence of said heat shock protein fragment, then a heat shock protein fragment capable of inducing an HSP-CD36 mediated process is identified.

64. The method of claim 63 wherein CD36 activity measured is the ability to interact with the heat shock protein fragment.

65. The method of claim 63 wherein the heat shock protein fragment is non-covalently associated with an antigenic peptide and CD36 activity measured is the ability to stimulate signal transducer activity.

66. The method of claim 63 wherein the heat shock protein fragment is non-covalently associated with an antigenic peptide and CD36 activity measured is the ability to stimulate a cytotoxic T cell response against the antigenic peptide.

67. A method for identifying a molecule that binds specifically to CD36, said method comprising:
(a) contacting CD36 with one or more test molecules under conditions conducive to binding; and
(b) identifying one or more test molecules that specifically bind to CD36.

68. The method of claim 67 wherein said test molecules are potential immunotherapeutic drugs.

69. A method for screening for molecules that specifically bind to CD36 comprising:
(a) contacting CD36 with one or more test molecules under conditions conducive to binding; and
(b) determining whether any of said test molecules specifically bind to CD36.

70. A method for identifying a compound that modulates the binding of a CD36 ligand to CD36 comprising:
(a) contacting CD36 with a CD36 ligand, or a CD36-binding fragment, analog, derivative or mimetic thereof, in the presence of one or more test compounds; and
(b) measuring the amount of CD36 ligand, or fragment, analog, derivative or mimetic thereof, bound to CD36,
such that if the amount of bound CD36 ligand measured in (b) differs from the amount of bound CD36 measured in the absence of the test compound, then a compound that modulates the binding of CD36 ligand to CD36 is identified.

71. The method of claim 70 wherein CD36 contacted in step (a) is on a cell surface.

72. The method of claim 70 wherein CD36 is immobilized to a solid surface.

73. A method for identifying a compound that modulates the interaction between CD36 and a CD36 ligand, comprising:
(a) contacting CD36 with one or more test compounds; and
(b) measuring the level of CD36 activity or expression,
such that if the level of activity or expression measured in (b) differs from the level of activity in the absence of one or more test compounds, then a compound that modulates the interaction between CD36 and a CD36 ligand is identified.

74. A method for identifying a compound that modulates signal transduction by CD36-expressing cells comprising:
(a) adding one or more test compounds to a mixture of CD36-expressing cells and a complex comprising a CD36 ligand and an antigenic molecule, under conditions conducive to CD36-mediated signal transduction;
(b) measuring the level of stimulation of antigen-specific cytotoxic T cells by CD36-expressing cells,
such that if the level measured in (b) differs from the level of said stimulation in the absence of the one or more test compounds, then a compound that modulates signal transduction by CD36-expressing cells is identified.

75. A method for modulating an immune response comprising administering to a mammal a purified compound that binds to CD36, in an amount effective to modulate an immune response in the mammal.

76. A method for treating or preventing a disease or disorder comprising administering to a mammal a purified compound that binds to CD36, in an amount effective to treat or prevent the disease or disorder in the mammal.

77. The method of claim 76 wherein the disease or disorder is cancer or an infectious disease.

78. A method for treating an autoimmune disorder comprising administering to a mammal in need of such treatment a purified compound that binds to CD36, in an amount effective to treat an autoimmune disorder in the mammal.


SEQUENCE LISTING (2 sequences)

SEQ ID NO: 1 - DNA, 1820 bp, Homo Sapiens
ggggatgcaactaagttgctgagacaagggaagagagatgaggaaccagagcttgtagaaaccactttaatcatatccaggagtttgcaagaaacaggtgcttaacactaattcacctcctgaacaagaaaaatgggctgtgaccggaactgtgggctcatcgctggggctgtcattggtgctgtcctggctgtgtttggaggtattctaatgccagttggagacctgcttatccagaagacaattaaaaagcaagttgtcctcgaagaaggtacaattgcttttaaaaattgggttaaaacaggcacagaagtttacagacagttttggatctttgatgtgcaaaatccacaggaagtgatgatgaacagcagcaacattcaagttaagcaaagaggtccttatacgtacagagttcgttttctagccaaggaaaatgtaacccaggacgctgaggacaacacagtctctttcctgcagcccaatggtgccatcttcgaaccttcactatcagttggaacagaggctgacaacttcacagttctcaatctggctgtggcagctgcatcccatatctatcaaaatcaatttgttcaaatgatcctcaattcacttattaacaagtcaaaatcttctatgttccaagtcagaactttgagagaactgttatggggctatagggatccatttttgagtttggttccgtaccctgttactactacagttggtctgttttatccttacaacaatactgcagatggagtttataaagttttcaatggaaaagataacataagtaaagttgccataatcgacacatataaaggtaaaaggaatctgtcctattgggaaagtcactgcgacatgattaatggtacagatgcagcctcatttccaccttttgttgagaaaagccaggtattgcagttcttttcttctgatatttgcaggtcaatctatgctgtatttgaatccgacgttaatctgaaaggaatccctgtgtatagattcgttcttccatccaaggcctttgcctctccagttgaaaacccagacaactattgtttctgcacagaaaaaattatctcaaaaaattgtacatcatatggtgtgctagacatcagcaaatgcaaagaagggagacctgtgtacatttcacttcctcattttctgtatgcaagtcctgatgtttcagaacctattgatggattaaacccaaatgaagaagaacataggacatacttggatattcaacctataactggattcactttacaatttgcaaaacggctgcaggtcaacctattggtcaagccatcagaaaaaattcaagtattaaagaatctgaagaggaactatattgtgcctattctttggcttaatgagactgggaccattggtgatgagaaggcaaacatgttcagaagtcaagtaactggaaaaataaacctccttggcctgatagaaatgatcttactcagtgttggtgtggtgatgtttgttgcttttatgatttcatattgtgcatgcagatcgaaaacaataaaataagtatgtaccaaaaaatattgcttcaataatattagcttatatattacttgttttcactttatcaaagagaagttacatattaggccatatatatttctagacatgtctagccactgatcatttttaaatataggtaaataaacctataaatattatcacgcagatcactaaagtatatctttaattctgggagaaatgagataaaagatgtacttgtgaccattgtaacaatagcacaataaagcactgtgccaaagttgtccaaaaaa

SEQ ID NO: 2 - protein, 472 aa, Homo Sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIQPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK